(19) United States
(12) Patent Application Publication
(10) Pub. No.: US 2022/0277859 A1
(43) Pub. Date: Sep. 1, 2022

(54) METHOD AND SYSTEM TO DIFFERENTIATE THROUGH BILEVEL OPTIMIZATION PROBLEMS USING MACHINE LEARNING

(71) Applicant: NEC Laboratories Europe GmbH, Heidelberg (DE)

(72) Inventor: Francesco Alesiani, Heidelberg (DE)

(21) Appl. No.: 17/361,339

(22) Filed: Jun. 29, 2021

Related U.S. Application Data
(60) Provisional application No. 63/151,072, filed on Feb. 19, 2021.

(51) Int. Cl. G16H 70/40; G06N 3/08; G06K 9/62

(52) U.S. Cl. CPC G16H 70/40; G06N 3/082; G06K 9/6269

(57) ABSTRACT

The present invention provides a method for bilevel optimization using machine learning. The method comprises: obtaining input data associated with the bilevel optimization; determining a solution for the bilevel problem; updating, based on the solution for the bilevel problem, a neural network using one or more intermediate parameters associated with the neural network and the bilevel optimization, wherein the one or more intermediate parameters are based on first output from the neural network and second output from a loss function associated with the neural network, wherein the first output is generated based on inputting the input data into the neural network; and outputting one or more finalized parameters for the bilevel optimization based on a change of the one or more intermediate parameters reaching a pre-determined threshold.

### CROSS-REFERENCE TO PRIOR APPLICATION

Priority is claimed to U.S. Provisional Application No. 63/151,072 filed on Feb. 19, 2021, the entire contents of which is hereby incorporated by reference herein.

### FIELD

The present invention relates to a method to differentiate through bilevel optimization problems in machine learning and application in bioinformatics and digital twin systems.

### BACKGROUND

Bilevel optimization is used to solve real world applications, such as price allocation or robust optimization for vehicle routing problems. Other example applications are:

1. Toll Setting Problem

2. Environmental Economics

3. Chemical Industry

4. Optimal design

5. Defense applications

6. Facility Location

7. Inverse Optimal control

8. Machine Learning

9. Economics/Principal-agent problems

10. Wildfire Emergency Response Optimization.

See, e.g., Sinha, Ankur, Pekka Malo, and Kalyanmoy, Dec. 2020, “A Review on Bilevel Optimization: From Classical to Evolutionary Approaches and Applications,” ArXiv:1705.06270 (the entire contents of which are hereby incorporated by reference herein).

In drug development, as an example, a problem is to evaluate the effect of the interaction of molecules (e.g., proteins) and cells or other genetic material. The process can be considered as solving an optimization problem. When the interaction or simulation considers antagonist interactions of elements, as for example the effect of anticorps, this optimization problem is a bilevel optimization, where the cell reacts to the solution of the proteins' interactions.

In drug discovery, it is desired to predict the effect of proteins. In this context, the cell reacts to the introduction of the new molecule or protein. It is desired to include this process inside a data-driven machine learning system, so that the parameters of the system may be trained and learned from the observational measures of the interactions and the output.

Sometimes, the interaction is captured by the gene regulatory network, but this description is static and may not capture more complex interactions. So, it might not be able to predict correctly the effect in new conditions. The present inventors have recognized that integration of the interactions has the potential of giving a more realistic solution of the problem, and thus, higher accuracy and better drug development.

Further, an optimization process that selects the more suited molecule for a specific effect may be used, but this procedure may have parameters that are not known. There is no clear way of including these nested optimization problems inside a machine learning system.

As another example, in hospital digital twin (e.g. for scheduling operations), there may be a system that simulates the functioning of the whole or part of a hospital. This system may include solutions of optimization problems and there is a bilevel problem when these problems are nested. For example, the health and recovery of the patient may be described as a simulation or a solution of a dynamic problem, while the intervention of the hospital or subpart of it, may be described as a control mechanism or a solution of a linked optimization problem.

Another example may be the sub-system that assigns the operation rooms, the operation teams and the patients. This problem depends on the recovering of the patient, which on the other hand depends on some other dynamics whose parameters are not known.

The inventors have recognized that there may be questions as to how it is possible to learn such dynamics, or how to optimize the allocation which depends on the patient recovery dynamics.

### SUMMARY

In an embodiment, the present invention provides a method for solving bilevel optimization using machine learning. The method comprises: obtaining input data associated with the bilevel optimization; determining a solution for the bilevel problem; updating, based on the solution for the bilevel problem, a neural network using one or more intermediate parameters associated with the neural network and the bilevel optimization, wherein the one or more intermediate parameters are based on first output from the neural network and second output from a loss function associated with the neural network, wherein the first output is generated based on inputting the input data into the neural network; and outputting one or more finalized parameters for the bilevel optimization based on a change of the one or more intermediate parameters reaching a pre-determined threshold.

### BRIEF DESCRIPTION OF THE DRAWINGS

Embodiments of the present invention will be described in even greater detail below based on the exemplary figures. The present invention is not limited to the exemplary embodiments. All features described and/or illustrated herein can be used alone or combined in different combinations in embodiments of the present invention. The features and advantages of various embodiments of the present invention will become apparent by reading the following detailed description with reference to the attached drawings which illustrate the following:

FIG. 1 shows a flow of information for an exemplary processor for using end-to-end ML datasets for bilevel optimization according to one or more embodiments of the present invention;

FIG. 2 shows an example of the processor having two sub-processors for using end-to-end ML datasets for bilevel optimization according to one or more embodiments of the present invention;

FIG. 3 illustrates an embodiment of a machine learning system;

FIG. 4 shows a machine learning system where the function includes a bi-level optimization problem;

FIG. 5 shows a bi-level problem as a component of a differentiable system;

FIG. 6 shows a bilevel optimization problem of a differentiable system;

FIG. 7 shows a division of the forward and backward steps using the bilevel optimization processor according to one or more embodiments of the present invention;

FIG. 8 shows the bilevel optimization processor implementing bilevel optimization with discrete variable(s) using end-to-end machine learning according to one or more embodiments of the present invention;

FIG. 9 illustrates an embodiment of the present invention having a digital twin of a smart hospital;

FIG. 10 illustrates a drug development flow: from training to testing and selection of candidates;

FIG. 11 illustrates an embodiment that solves a logistics application using bilevel optimization and a neural network;

FIG. 12 illustrates an embodiment for optimization of the response to natural or dangerous events using bilevel optimization and a neural network;

FIG. 13 illustrates an embodiment for industrial optimization using bilevel optimization and a neural network;

FIG. 14 illustrates an embodiment for smart transportation using bilevel optimization and a neural network;

FIG. 15 illustrates an embodiment for market simulation using bilevel optimization and a neural network;

FIG. 16 illustrates an example of an initial configuration of orthogonal vectors in 3D;

FIG. 17 illustrates an output after allying an embodiment of the method of the present invention;

FIG. 18 shows an exemplary diagram for performing a task of learning a system, where x and y are the solutions of a bilevel optimization problem;

FIG. 19 shows an example of an application to model a dynamical system with a control neural network where the interaction between the controller and the process is modelled as the solution of a bilevel optimization problem;

FIG. 20 shows a visualization of an optimal control learning network;

FIG. 21 shows a table indicating the optimal control average cost and a comparison of the training performance;

FIG. 22 shows an example of the shortest path in a WARCRAFT II tile set;

FIGS. 23a and 23b show an example of the shortest path without and with interdiction; and

FIG. 24 shows a discrete bilevel variables and dependence diagram.

### DETAILED DESCRIPTION

The inventors have recognized that if one were able to describe and include problems such as those described above (and similar) inside a learnable system, the abundant data from real systems (e.g., hospitals) can be used, and the missing parameters that govern the various dynamics may be learned. In other words, as will be described in further detail below, the present invention describes using data from real systems to train a machine learning system for bi-level optimization problems. Thus, this may enable the change of the functioning of the real systems (e.g., hospitals) in a way that is beneficial for the relevant actors (e.g., patients, personnel of the hospital, and for the hospital as a whole).

Traditionally, various optimization solvers have been designed to provide exact or approximate solutions to such problems. For example, branch-and-bound or branch-and-cut methods have been developed to solve discrete optimization problems. Convex optimization tools may be used to solve convex problems, based on primal-dual optimization. The family of evolutionary methods, such as stochastic annealing or genetic algorithm, may be used to solve discrete optimization when search space is too large and approximate solutions and easy to develop solution is required.

Despite the progress in optimization solvers, currently, it is not known in the art how such a method could be integrated for end-to-end machine learning systems (e.g., systems that operate machine learning algorithms and/or neural networks). A basic approach may be to implement the algorithm in some differential programming language (e.g., JAX (JAVA application programming interface (API) for extensible markup language (XML)) or Autograd) and let the language compute the gradient necessary to use the method in an end-to-end (e.g., differentiable) learning process. JAX and Autograd are libraries that allow for implementation of auto differentiation of functions and are used within the present invention. However, solely using a differential programming language with a library, such as JAX, may have two main drawbacks: 1) a need to re-implement the method with the risk that some function or library is not available; 2) uncontrolled memory usage. In other words, the memory is the most limiting factor, indeed the auto differentiation language needs to store all the intermediate results and it may be incapable of scaling with large amount of data or long iterations, which may be required for the end-to-end machine learning systems. To put it another way, coding the algorithm such that it solves the problem with differentiable operations and then computes the gradient is possible, but it may lead to a very large memory usage and may also have stability issues such as rounding errors. Accordingly, the present invention may substitute the normal execution flow of the library (e.g., the JAX and/or Autograd) and compute the gradients instead of permitting the libraries to do it blindly.

To put it another way, the present invention may reduce the memory usage, which may be advantageous for an end-to-end learning system because of at least the two following reasons. First, implementing the solver directly with differentiable operators may be a complex task in its own (e.g., not all operations may be easily reduced to differentiable operations) as mainly, when the algorithm runs, it may create a very long temporal unroll of the operations that creates a very long relationship (e.g., chain of operations) that may require large amounts of memory, which is a critical resource (e.g., graphics processing units (GPU) memory is heavily restricted by hardware). Second, the present invention natively requires N\hat{ }2 memory size and with modern architecture (million if not billions of parameters) would not fit the memory. As such, by using the vector jacobian product (VJP), the present invention does not require the ability to materialize the entire jacobian (N\hat{ }2 entries), but may operate on N space (the number of variables), thus allowing the use in modern computing systems.

Embodiments of the present invention provide methods and systems to integrate an externally computed solution into an end-to-end differentiable learning process for bilevel optimization problems and/or applications. For instance, a bilevel optimization problem may be learned using one or more solvers. The systems and methods may learn parameters of the bilevel problem as a component of the end-to-end learning system. In some instances, the bilevel problem may be just one component of a system. In some examples, there may be multiple bilevel problems in a single system.

A method and system of the present invention enables including a bilevel optimization problem inside an end-to-end trainable machine learning system. In some instances, the data driven training of bilevel optimization may allow for modeling complex systems and training using data, without too limiting assumptions on the dynamics of the system. A method of the present invention allows for inclusion, for example, molecule and cellular interactions and molecule selection process for drug discovery and train from observational data. Similarly, a hospital digital twin can include unknown dynamics and solution of optimization problems, which may be now trained end-to-end. Additional embodiments of using the end-to-end trainable machine learning system for other systems such as robust vehicle routing applications, wildfire prevention and response or emergency management applications, and so on are further described in detail below. In other words, the present invention may describe using end-to-end trainable machine learning systems and methods for bilevel optimization problems for a variety of applications and/or uses.

To put it another way, the method and system of the present invention may solve bilevel optimization problems using end-to-end trainable machine learning systems by using several phases. In the first phase, the system may collect relevant data and perform preprocessing. In the second phase, the system may determine a description of the problem with a combination of a machine learning (ML) model or dataset (e.g., a neural network dataset) and a (bilevel) optimization problem. In the third phase, the system may train the model with the available data. In the fourth phase, the system may use the model for selection of potential candidates based on new targets that meet the minimum requirements. In the fifth phase, the system may perform synthesis and evaluation of the candidates, with possible iterations of the process.

In an embodiment, the present invention provides a method for bilevel optimization using machine learning. The method comprising: obtaining input data associated with the bilevel optimization; determining a solution for a bilevel problem; updating, based on the solution for the bilevel problem, a neural network using one or more intermediate parameters associated with the neural network and the bilevel optimization, wherein the one or more intermediate parameters are based on first output from the neural network and second output from a loss function associated with the neural network, wherein the first output is generated based on inputting the input data into the neural network; and outputting one or more finalized parameters for the bilevel optimization based on a change of the one or more intermediate parameters reaching a pre-determined threshold.

In an embodiment, determining the solution for the bilevel problem is based on using Karush-Kuhn-Tucker (KKT) reformulation of the bilevel problem and/or alternating direction method of multipliers (ADMM).

In an embodiment, updating the neural network using the one or more intermediate parameters comprises: determining one or more gradients based on the second output from the loss function; and updating the neural network based on providing the one or more gradients to the neural network.

In an embodiment, determining the one or more gradients is further based on an implicit theorem of differentiability and/or Karush-Kuhn-Tucker (KKT) optimality conditions.

In an embodiment, determining the solution for the bilevel problem comprises determining one or more first intermediate parameters to the bilevel optimization based on the first output from the neural network, and wherein updating the neural network using the one or more intermediate parameters comprises: inputting the one or more first intermediate parameters into the loss function to determine the second output; determining one or more second intermediate parameters based on the second output, wherein the one or more second intermediate parameters are associated with one or more gradients; and providing the one or more second intermediate parameters to the neural network to update the neural network.

In an embodiment, the method further comprises: repeatedly determining the one or more first intermediate parameters and updating the neural network using the one or more intermediate parameters; after each iteration of updating the neural network, comparing a change of the one or more intermediate parameters with the pre-determined threshold; and based on the change of the one or more intermediate parameters reaching the pre-determined threshold, generating the one or more finalized parameters using the one or more first or second intermediate parameters associated with a last iteration of updating the neural network.

In an embodiment, repeatedly determining the one or more first intermediate parameters and updating the neural network using the one or more intermediate parameters is based on the change of the one or more intermediate parameters not reaching the pre-determined threshold.

In an embodiment, the one or more first intermediate parameters to the bilevel optimization are continuous variables.

In an embodiment, the one or more first intermediate parameters to the bilevel optimization are discrete variables.

In an embodiment, determining the one or more second intermediate parameters associated with the one or more gradients is based on vector jacobian product (VJP) or jacobian vector product (JVP).

In an embodiment, the bilevel problem is a hospital digital twin of a smart hospital, and wherein the method further comprises: using the one or more finalized parameters to design a new system for the bilevel problem, wherein the new system optimizes patient scheduling for the smart hospital, room scheduling for the smart hospital, personnel scheduling for the smart hospital, recovery dynamic for the smart hospital, or procurement for the smart hospital.

In an embodiment, the bilevel problem is a drug development problem, and wherein the method further comprises: using the one or more finalized parameters to design a new system for the bilevel problem, wherein the new system indicates drug candidates for molecule synthesis.

In another embodiment, the present invention provides a system comprising one or more processors which, alone or in combination, are configured to provide for execution of a method comprising: obtaining input data associated with bilevel optimization; determining a solution for the bilevel problem; updating, based on the solution for the bilevel problem, a neural network using one or more intermediate parameters associated with the neural network and the bilevel optimization, wherein the one or more intermediate parameters are based on first output from the neural network and second output from a loss function associated with the neural network, wherein the first output is generated based on inputting the input data into the neural network; and outputting one or more finalized parameters for the bilevel optimization based on a change of the one or more intermediate parameters reaching a pre-determined threshold.

In an embodiment, updating the neural network using the one or more intermediate parameters comprises: determining one or more gradients based on the second output from the loss function; and updating the neural network based on providing the one or more gradients to the neural network.

In a further embodiment, the present invention provides a tangible, non-transitory computer-readable medium having instructions thereon which, upon being executed by one or more processors, alone or in combination, provide for execution of a method comprising: obtaining input data associated with bilevel optimization; determining a solution for a bilevel problem; updating, based on the solution for the bilevel problem, a neural network using one or more intermediate parameters associated with the neural network and the bilevel optimization, wherein the one or more intermediate parameters are based on first output from the neural network and second output from a loss function associated with the neural network, wherein the first output is generated based on inputting the input data into the neural network; and outputting one or more finalized parameters for the bilevel optimization based on a change of the one or more intermediate parameters reaching a pre-determined threshold.

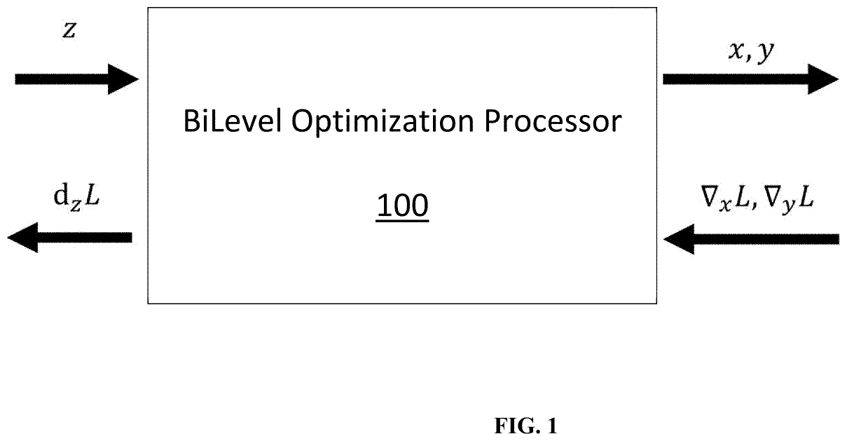

FIG. 1 shows a flow of information for an exemplary processor 100 for using end-to-end ML datasets for bilevel optimization according to one or more embodiments of the present invention.

As illustrated, an embodiment of the system of the present invention may be composed of a processor 100 that receives input values z, ∇ₓL, ∇_{y}L and produces output values x, y, d_{z}L. The processor 100 is and/or includes a central processing unit (CPU), controller, logic, module, engine, and/or other hardware/software that is configured to perform the functionalities described herein such as using end-to-end ML datasets for bilevel optimization.

The processor 100 is not constrained to any particular hardware, and the processor's configuration may be implemented by any kind of programming (e.g., embedded Linux) or hardware design—or a combination of both. For instance, the processor 100 may be formed by a single processor and/or controller, such as general purpose processor with the corresponding software implementing the described control operations. On the other hand, the processor 100 may be implemented by a specialized hardware, such as an ASIC (application-specific integrated circuit), an FPGA (field-programmable gate array), a DSP (digital signal processor), or the like. In some instances, the processor 100 may be implemented as engines, software functions, modules, and/or applications. In other words, the functionalities of the processor 100 may be implemented as software instructions stored in memory and executed by one or more hardware processors such as CPUs.

Referring to FIG. 1, the variable z represents the parameters of the optimization problem such as the differential parameters of a neural network (e.g., output from a neural network and/or a machine learning dataset). The variables x and y represent the solution of the optimization problem. For instance, the optimization problem may be a bilevel optimization problem. The x variable represents the outer level variables or the leader variables (e.g., the variables of the controller and/or processor). The y variable represents the solution of the follower or the inner problem and may be defined once the x variable is defined. The x and y variables may then be used in further stages of the system. In other words, the z represents the input to the bi-level optimization processor 100 and x and y are the outputs of the processor 100. The ∇ₓL and ∇ₓL are loss function (L) gradients associated with the variables x and y (which is given). The d_{z}L is a derivative of the loss function associated with the variable z (which may need to be estimated).

The functionalities of the processor 100 as well as the definitions for the input/output values (e.g., input values z, ∇ₓL, ∇_{y}L and output values x, y, d_{z}L) will be described in further detail below.

In some instances, the processor 100 may include two or more sub-processors. The first sub-processor may be configured to be a solver of one or more bilevel optimization problems. The second sub-processor may be configured to be an estimator of the gradient around the current computed solutions of the one or more bilevel optimization problems. In some instances, the sub-processors may be separate hardware components that are configured to perform the above-mentioned functionalities. In other instances, the sub-processors may be implemented as sub-modules and/or sub-engines (e.g., software instructions stored in memory).

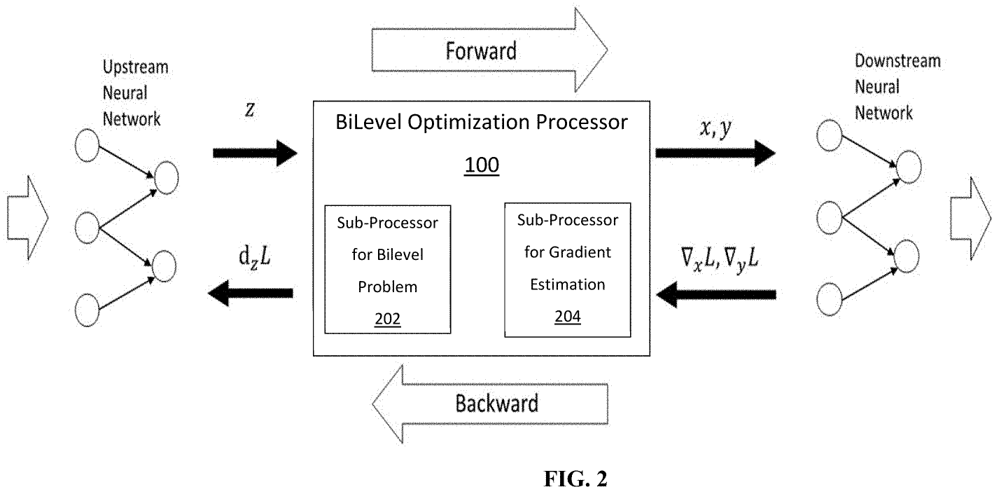

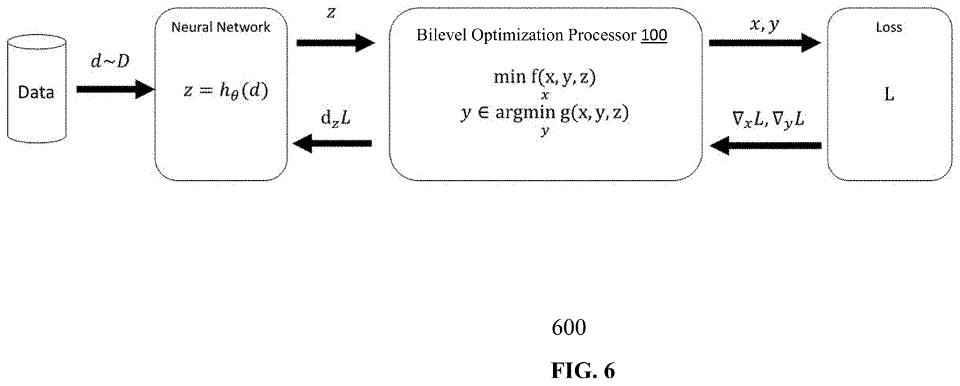

FIG. 2 shows an example of the processor 100 having two sub-processors for using end-to-end ML datasets for bilevel optimization according to one or more embodiments of the present invention. For example, FIG. 2 illustrates an approach of the method, which includes separating the computation of the solution of the bilevel problem and the computation of its gradient around the computed solution into two separate sub-processors 202 and 204. For instance, sub-processor 202 may be for solving the bilevel problem (e.g., determining and/or computing optimizations for a bilevel application). Sub-processor 204 may be for gradient estimation (e.g., determine gradient estimators around current solution). In some instances, the bilevel optimization processor 100 may be used for training the neural networks. In such instances, a forward pass (e.g., inputting the variables z into the processor 100 to determine the variables x and y) and a backward pass (e.g., based on the variables x and y, the downstream neural network may determine the gradients and feed them back into the processor 100). The gradients are used to determine the accuracy of the system as well as to further update the weights of the neural network to further improve the accuracy of the system. In some examples, a loss function may be used to replace and/or may be used in conjunction with the downstream neural network. Embodiments describing using the loss function are shown and described below (e.g., see FIG. 6). In some variations, the bilevel optimization processor 100 shown in FIG. 2 may be used during deployment. In other words, after training the neural networks, the bilevel optimization processor 100 may determine solutions (e.g., the variables x and y) for the bilevel optimization problem. In such variations, only the forward pass may be used (e.g., inputting z to determine x and y) and the backward pass may be omitted. Additionally, and/or alternatively, in self-training or online training, the neural networks may be continuously updated and may use the backward pass even during deployment (e.g., using the gradients of the loss functions to continuously update the neural networks).

In operation, data may be input into the upstream neural network and the output of the upstream neural network, z, may be provided to the bilevel optimization processor 100. The output of the bilevel optimization processor 100 may be x and y, which may be provided to the downstream neural network. These may be input into the downstream neural network, which may generate the outputs such as ∇ₓL, ∇_{y}L. These outputs may be put back into the bilevel optimization processor 100, which may produce output value(s) such as d_{z}L that are fed back into the upstream neural network.

In some instances, the sub-processor 202 for the bilevel problem may obtain z form the upstream neural network and generate the output x and y that are provided the downstream neural network. The sub-processor 204 for the gradient estimation may obtain the outputs of the downstream neural network (∇ₓL, ∇_{y}L) and generate the output d_{z}L that is provided to the upstream neural network. This will be explained in further detail below.

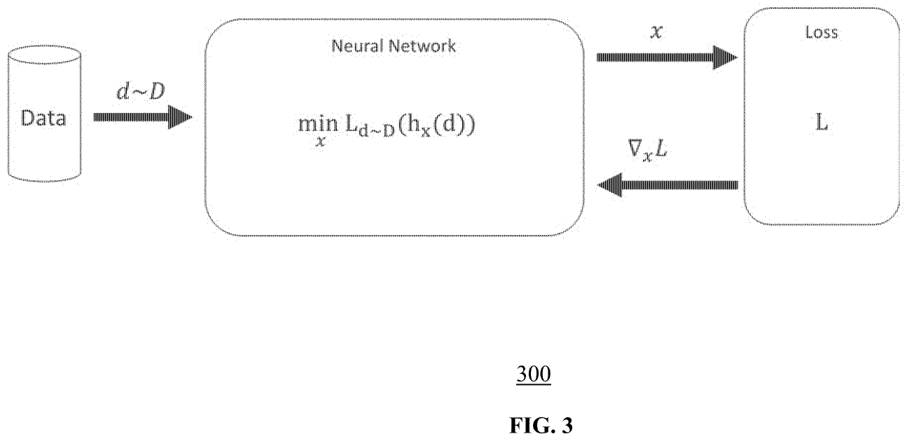

FIG. 3 illustrates a modern machine learning system 300. The modern ML system 300 includes: 1) Input data D; 2) A function hₓ (d) parametrized by some parameter x; and 3) A loss function L that describes the error in using the function f instead of the original data. In other words, the ML system 300 may be composed of and/or include (D, h, L) or the input data D, the function hₓ, and the loss function L. As shown, d˜D indicates that the training samples “d” is taken from the total sample data “D” and the result may be the mean over these samples.

Referring to the system 300, the function h is differentiable and it is possible to propagate the error of the prediction of d on the data D via the loss function L back to the parameter of the neural network. Therefore, by using this, the system 300 may be able to update the parameter x to optimize the loss function. In other words, system 300 may be a generic system where h is the function that maps the input to d to some output that is evaluated over the loss L and depends on a variable x, which is minimized. In system 300, x may be the neural network parameter, h is the neural network, and d is the input/output data.

However, for bilevel optimization problems, the parameters and functions may directly relate to further nested parameters and/or functions, which causes difficulty for the modern machine learning system 300 to optimize the loss function by updating the parameters. For instance, a neural network may only provide a single level solution that is unable to generalize the bilevel problem (e.g., it provides only variables x, but not both x and y that represent the leader and follower solutions). Similarly, if a sequential problem is used, this may lose performance (e.g., accuracy) as the decisions may be taken prior to determining and seeing the consequences. Accordingly, as will be explained below, the present invention uses the processor 100 with machine learning (e.g., neural networks) for bilevel optimization problems. For instance, a processor (e.g., processor 100) may perform one or more optimization tasks as specification of a function (e.g., function h) such that the processor may solve the bilevel optimization problem.

The bilevel optimization problem may be characterized by:
1. An outer problem or leader and its objective function f(x, y, z) where x, y, z are the parameters of the outer problem. The outer problem may be solved in the parameter x; however, parameter y may depend on the inner problem solution;
2. An inner problem or follower, specified by a function g(x, y, z) where x, y, z are parameters of the inner problem. The inner problem is solved in the parameter y; and
3. The parameter z, which may be some external parameter(s) that specify the function f and g and that may depend on (e.g., based on) at least a portion (e.g., some) of the external data d˜D.

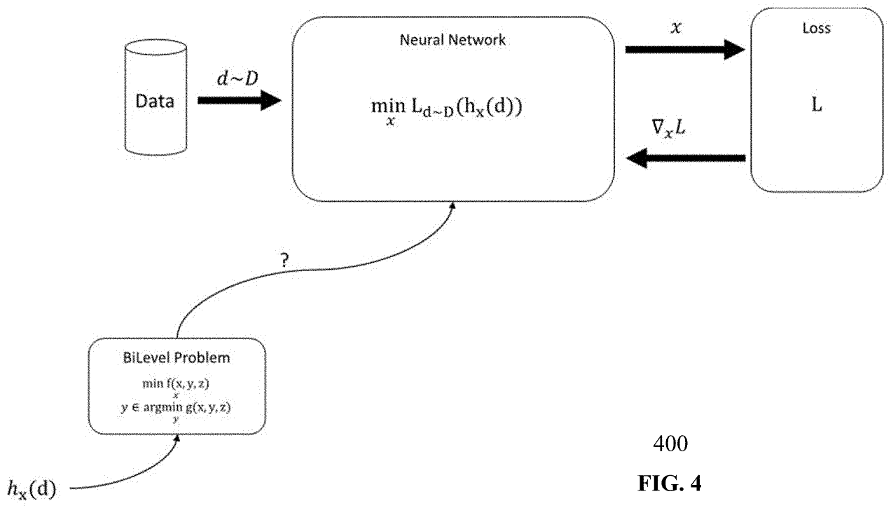

FIG. 4 shows a machine learning system 400 where the function includes a bi-level optimization problem. The top portion of FIG. 4 may be similar to the machine learning system 300 of FIG. 3 as there is external data D, a loss function L, and the neural network. However, FIG. 4 may further include incorporating a bilevel problem into the machine learning system 300. For instance, the bilevel problem may be characterized by the outer problem with its objection function f(x, y, z) and the inner problem with its function g(x, y, z). The “?” denotes the idea that it is unclear how to solve a bilevel problem with a current single level method (e.g., the traditional machine learning system 300).

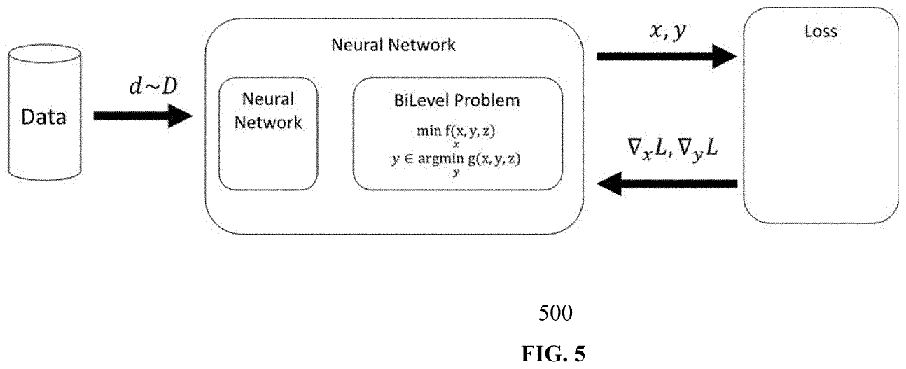

FIG. 5 shows a bi-level problem as a component of a differentiable system 500. In an exemplary case, z is the output of a neural network and defines the bi-level problem (e.g., the bi-level problem of FIG. 4). When a bilevel problem is integrated into a differentiable system 500, there are two output variables x and y as the output, the outer and inner variables (e.g., the outer variable y and the inner variable x). For example, y may be the solution of a buying problem and x may be the price assigned to the goods.

FIG. 6 shows a bilevel optimization problem of a differentiable system 600. The differential system 600 includes a processor (e.g., processor 100) that may be used to solve the bilevel optimization problem using machine learning. In other words, the differentiable system 600 includes the bilevel optimization processor 100 of FIG. 1, which may be used for adapting end-to-end ML datasets (e.g., the neural network/neural network dataset) for bilevel optimization. Here, the input of the bilevel optimization processor 100 is the variable z, which depends on the external data, while the output of the processor 100 are the variable x, y. To put it another way, the external data D may be input to the neural network and the output of the neural network z may be input to the bilevel optimization processor 100. In order to update the parameter of the function z=h_{θ}(d), the variation of the parameter z with respect of the loss function, d_{z}L or the total variation, may need to be determined by the bilevel optimization processor 100. In other words, in the upstream neural network, the output may be z. Further, the bilevel problem may use z to parametrize the bilevel problem (e.g., z may be the weights of the neural network that specify the cost of the problem). For instance, in an embodiment, z may be the variable indicating a cost of traversing a pixel. The input may be the image and the cost may be computed from the image using a neural network of parameter theta (θ). The bilevel problem to be solve is to find the shortest path, which is the variable y with an adversarial variable x.

With the loss function, the bilevel optimization processor 100 may determine (e.g., compute) the gradient with respect to x and y variables: ∇ₓL, ∇_{y}L. The bilevel optimization processor 100 may then determine (e.g., compute) the mapping between these gradients and the total variation: (∇ₓL, ∇_{y}L)→d_{z}L, which is fed back into the neural network as the loss function.

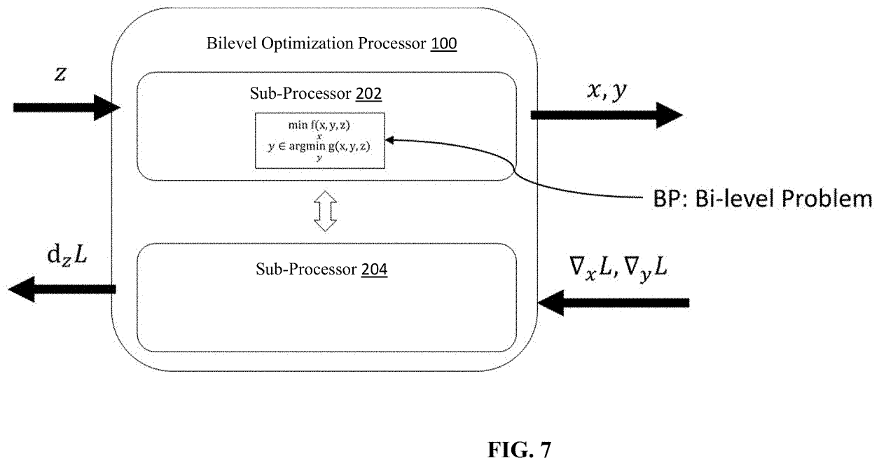

FIG. 7 shows a division of the forward and backward steps using the bilevel optimization processor 100. For instance, the processor 100 may include sub-processors 202 and 204 as described above in FIG. 2. The sub-processor 202 may be for solving the bilevel problem (e.g., determining and/or computing optimizations for a bilevel application) and the sub-processor 204 may be for gradient estimation (e.g., determine gradient estimators around current solution). In operation, for solving for the bilevel optimization, the bilevel optimization processor 100 may use:
1. An external library to compute the value of solution x, y given z (solution solver);
2. Another sub-processor (e.g., sub-processor 204) for computing the gradient, at the solved point x, y, z based on the shapes off and g (gradient solver).

The solution solver (e.g., the sub-processor 202) can be an existing solver. For instance, the solution solver/external library may obtain the specification for the bilevel optimization problem and the parameters of the bilevel optimization problem. Then, it may return a solution. For certain classes of problems, it is possible to formulate a single level problem. In other cases, it is transformed to a single level problem with an increasing number of constraints that account for the inner solution. The external library may internally use gradient computation to find local minima, but in general is not differentiable as solver. In some instances, to solve the bilevel problem, the KKT (karush-kuhn-tucker) reformulation of the bilevel problem may be used and solved using ADMM (alternating direction method of multipliers). For example, this “external” solver uses the KKT by computing the optimality conditions (KKT conditions) with a defined set of equation to solve such that the ADMM method may be applied to iteratively determine this solution. In some examples, this may only determine a local solution. ADMM is an iterative procedure where the main variable is duplicated for each constrains, and where solving the single constrain is simpler.

The gradient solver (e.g., the sub-processor 204) may use the implicit theorem of differentiability and the KKT optimality conditions to solve the continuous version and the random smoothness and linear function for the discrete case. For instance, the gradient solver computes or estimates the total gradient of the solution (e.g., the variables x and y) with respect to the output from the neural network (e.g., the variable z). This may be performed by defining a system of equations defined by the implicit function for the continuous case. Whereas for the discrete variable case, this may be performed by estimating the total gradient by propagating the gradient estimation from the inner level to the outer level. The estimation of the gradient may be based on the perturbation or change of variable principles. In other words, the gradient solver may use the implicit function gradient directly to iteratively improve the outer problem solution, which may be similar to gradient descent method. Otherwise, the gradient solver may use the stochastic gradient descent ascent method for minimum maximum (min-max) problems.

Continuous Variables: in some examples, both x and y variables may be considered as being continuous and/or are continuous, which is shown below:

$${min f ( x , y , z )}_{x}$$

$${y ∈ arg min g ( x , y , z )}_{y}$$

The solution solver (e.g., sub-processor 202) may be used to determine the optimal x, y variables, given z. Once these variables are determined, the gradients of the function f and g can be computed (e.g., by using the sub-processor 204). Using the KKT optimality conditions and the implicit theorem, the bilevel optimization processor 100 may construct the function that maps (∇ₓL, ∇_{y}L)→d_{z}L. The bilevel optimization processor 100 may use the vector jacobian product (VJP) operation and/or the jacobian vector product (JVP) operation to implement these operations efficiently in terms of memory. In particular, without using the vector jacobian product (VJP) or jacobian vector product (JVP) formulation, matrices of size m×n, n×p, and m x p may need to be operated upon, where n, m, p are the size of x, y, and z variables. In contrast, with VJP and/or JVP, the bilevel optimization processor 100 may operate with size n x 1, m x 1, and p x 1, which may save computational resources and/or memory.

VJP is vector-jacobian multiplication and JVP is the Jacobian-vector multiplication. The jacobian is a matrix and represents the change in the tangent vector induced by the function from the source space to the target space. VJP is used to compute the change in the source space that generates a specific change in the target space, while JVP is used to propagate a change in the source space to the target space. The VJP and JVP are shown and described in further detail below with respect to Eq. (4).

Linear Equality Constraints: In some instances, Ax=b or By=c equality may occur in the outer or inner problem. In both cases, there can be a change of variables:

x(r)=x₀+ [symbol] r|Ax₀=b

y(s)=yₒ+ [symbol] s|Bx₀=c

Referring to the above, x(r) and y(s) are new parametrization of the variables x and y such that these equations are now solving for r and s. A, b, B, and c define equality constraints within equations. The equality constraints may be re-parameterized and used similarly to the approaches described above.

Inequality Constraints: In some examples, a barrier method formulation may be used to deal with inequality constraints:

$$f ( x , y , z ) \to tf ( x , y , z ) - {\sum }_{i} ln ( - {f}_{i} ( x , y , z ) )$$

$$g ( x , y , z ) \to tg ( x , y , z ) - {\sum }_{i} ln ( - {g}_{i} ( x , y , z ) )$$

Referring to the above, t is a scalar parameter, gᵢ and fᵢ are user defined functions that define the inequality. The inequality constraints may be used to extend the present invention also in case of inequality.

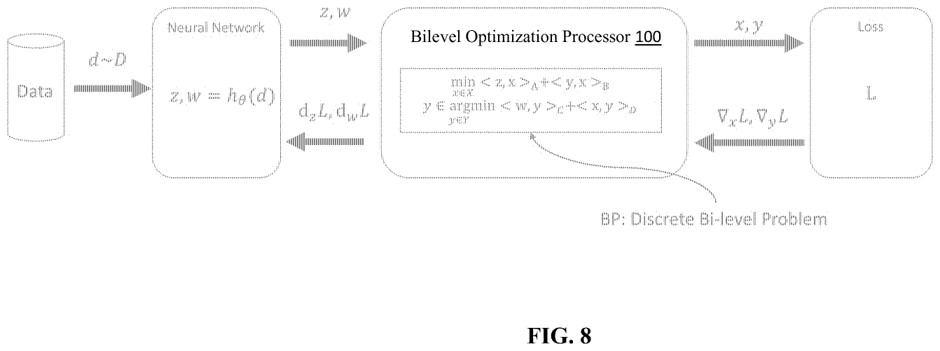

FIG. 8 shows the bilevel optimization processor 100 implementing bilevel optimization with discrete variable(s) using end-to-end machine learning according to one or more embodiments of the present invention. For example, the bilevel optimization processor 100 may optimize bilevel problems where the variables x and y are discrete. This may represented by the conditions x∈X and y∈Y, where X and Y are the domains of the two variables. <z, x>_{A} represents the generalized inner product, parametrized by the matrix A: <z, x>_{A}=z^{T}Ax.

In some variations, the bilevel optimization processor 100 may implement bilevel optimization with a mixed version where outer and inner problem use continuous and discrete variables.

Embodiment(s) for implicit differentiation and KKT conditions: In some instances, the bilevel optimization processor 100 may implement bilevel optimization for continual variables. For instance, the bilevel optimization processor 100 may use implicit differentiation to compute the gradients of variables and apply the KKT conditions to relate the variables.

Argmax (arguments of the maxima) embodiment(s): In some examples, the bilevel optimization processor 100 may use two tools (e.g., the argmax example and the smoothness operator/variable change) to deal with discrete variables. The first tool may be the argmax example. This states the following equality:

$${∇}_{z} {max}_{x} < z , x > = {x}^{*} ( z )$$

where

$${x}^{*} ( z ) = arg {max}_{x ∈ X} < z , x >$$

with <z, x> indicating the inner product and x* indicating the optimal value. When the linear mapping is used:

$${∇}_{z} arg {max}_{x} < z , x {>}_{A} = {Ax}^{*} ( z )$$

which may have the equality extended for the general case:

$${∇}_{z} {max}_{x ∈ X} σ ( < z , x > ) = σ ( < z , {x}^{*} > ) {x}^{*}$$

with σ(x) a non linear function. The value can be recovered as root point of:

$$σ ( < z , {x}^{*} > ) {x}^{*} σ ( < z , {x}^{*} > ) {x}^{*} - g = 0$$

$$where g = {∇}_{z} {max}_{x ∈ X} σ ( < z , x > ) .$$

Smoothness operator embodiment(s): The bilevel optimization processor 100 may use smoothness operator in order to compute an estimation of the gradient with discrete variables.

First, the solver function is defined:

$$Φ ( z ) = {min}_{x ∈ X} < z , x >$$

which maps the parameter z to the minimization value. The smoothness operator is defined as the evaluation of this function over a perturbed variable:

\tilde{Φ}_{μ}(z)=E_{u˜U}Φ(z+μu)

where u˜U is a random variable (of zero mean) and μ is a parameter.

In order to recover the optimal value, the following may be applied:

∇_{z}\tilde{φ}_{μ}(z)=x*(z).

As an example of a solution of the bilevel problem, the bilevel optimization processor 100 may use a generic solver for computing the solution of the bilevel problem.

The bilevel optimization processor 100 may use the KKT conditions and ADMM method to solve the bilevel problem for the continual variable case. For the discrete case, the bilevel optimization processor 100 may use a linear integer solver or a relaxation of the problem.

Memory Efficiency: a contributing point for improving memory efficiency is the use of VJP and/or JVP. For instance, in an embodiment, the present method and/or the bilevel optimization processor 100 may use vector jacobian product (VJP) or jacobian vector product (JVP) to compute the gradient for computational advantage.

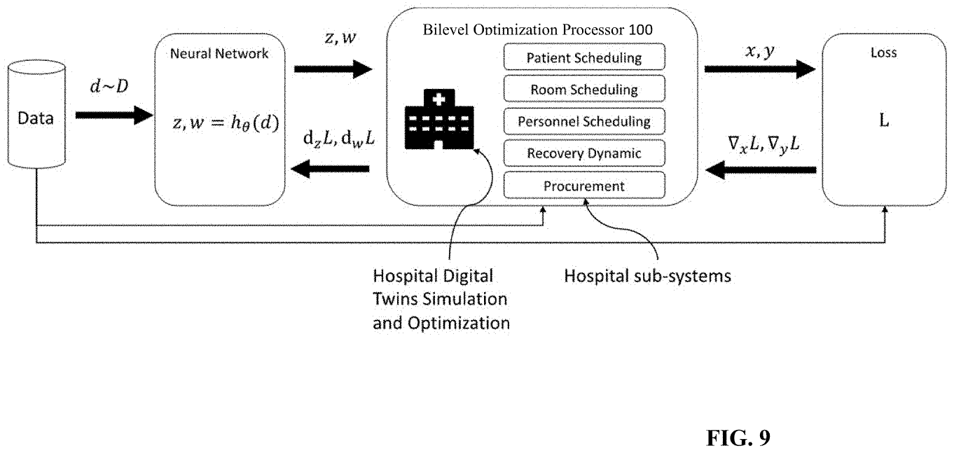

FIG. 9 illustrates an embodiment of the present invention having a digital twin of a smart hospital. A hospital digital twin may be used to replicate the status of the various systems relevant in the functioning of a hospital. These can be, for example:
1. The scheduling of the patient, from room where they stay or the room where an operation is performed;
2. The scheduling of personnel at any level, nurses, physicians, operation teams;
3. The scheduling of the rooms, including the preparation and cleaning phases;
4. The recovery of a patient, which may depend on the visit of personnel, the prescriptions and the operations;
5. The procurement of goods for the hospital, from the order to the delivery and disposal; and
6. The functioning of the hospital itself, like heating and disposal services.

The control of the hospital and the solution of the subsystems and bilevel problem are described herein. The bilevel optimization processor 100 may implement a model such that it is integrated and the parameters trained based on real data. Parameters can be in the form of electronic health record (EHR), but also from the ERP of the Hospital. The information may be stored in a clinical data repository/health data repository (CDHR); and thus may be typically available in a large number of hospitals. The ability to describe the hospital and a model inside a machine learning system allows for the evaluation of generic and specific parameters.

An example of EHR is fast healthcare interoperability resources (FHIR) and the type of information in EHR includes:
Administration data, which contain information to track patients, personnel, entities, devices, drugs;
Clinical data on the patients, including the reason to be in the hospital, condition at admission, and current state, preexisting pathologies, allergies, and process assigned for taking care of the patient;
Medications data;
Diagnostics data, which consist of observations, reports and conclusions;
Hospital workflow;
Financial data, from bills to funds; and
Clinical Monitoring and Decision Support.

Embodiments of the method of the present invention allow for the description of the interaction as a bilevel problem and for the integration of this problem as module and/or processor (e.g., processor 100) in machine learning. In a data driven way, the system is able to replicate the functioning of the hospital by back propagating the error in the prediction. Once the system is trained, it is possible to evaluate the function of the Hospital in alternative conditions, thus optimizing the entire or part of the Hospital in an automated way. This automatic decision process can be monitored by personnel during and post operation. For example, in an embodiment, the present invention may be used for the operation team and the patient to room allocation. For instance, the x variable may be the allocation to particular rooms while the y variable may be the cleaning of the operation room that cannot happen when the room is busy, but will also prevent the room from being used. In another embodiment, the present invention may be used for budget allocation to different departments, which has impact on the time operation to be performed. For instance, the operation may be modelled as an optimization problem with the variable y, while the budget allocation is the variable x.

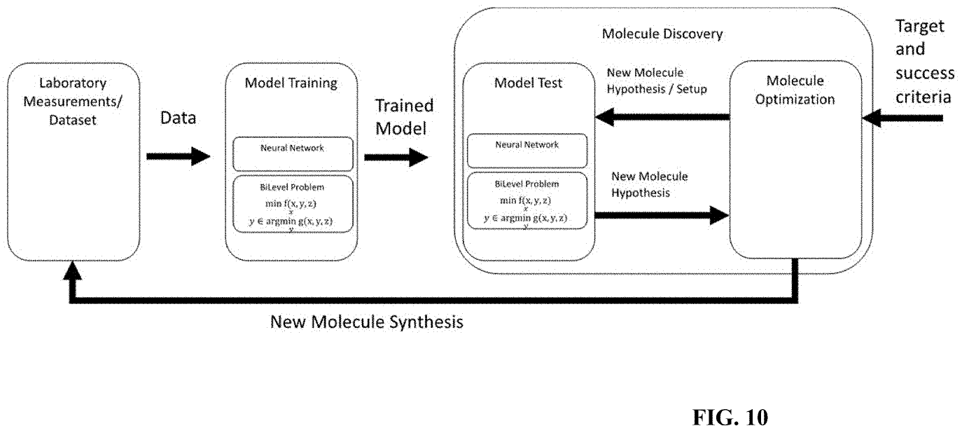

An embodiment of the present invention may be configured for drug development and molecule synthesis. For instance, in some examples, for drug development, the x variable may be the amount of protein injected into the cell while the variable y may be the process happening inside the cell. FIG. 10 illustrates an embodiment of a drug development flow, from training to testing and selection of candidates.

Drug development or molecule synthesis is based on predicting the effect of new molecule when interacting with a cellular environment or other biological entities. The interaction of a molecule or protein is a complex process that triggers the reaction of other elements in the biological system. One way to model this system is to consider that each element in the system as optimizing its own objective function or solving a separate optimization problem. All these problems are connected, bilevel problems model two optimization problems that are connected. The biological system may thus be described via a bilevel optimization problem, i.e. pair of connected problems.

In other stages of the drug development or molecule synthesis, an optimization problem may be solved to decide the optimal configuration. In this scenario, sub-problems describing another phase of the process that depends on the upper level decision may be present.

The overall process of discovery may thus include one more nested optimization problems to be trained from observational data. In a drug discovery process, the laboratory measurement may be an input. This data may be given as input to the training procedure whose output shall predict existing known positive and negative effects.

The inputs to the model may include:
1. The dataset or laboratory data, this includes the inputs and outputs, as the main effects and side effects; Data inputs are the laboratory setup: cells, disease, genes, proteins, processes;
2. Identification of the target, for example a protein or a gene that plays a significant role in the biology process or disease;
3. Description of the compound generation process, this process is an optimization problem parametrized by the input and target; this process also involves some unknown interaction mechanism that may be computed from the training data; and
4. The success criteria and their evaluations.

The overall process may include and/or the processor 100 may perform the following phases:
1. Collection of relevant data and preprocessing;
2. Description of the problem with a combination of neural network and (bilevel) optimization problems;
3. Training of the model with the available data;
4. Use of the model for selection of potential candidates based on new target which meet the minimum requirements; and
5. Synthesis and evaluation of the candidates, with possible iteration of the process

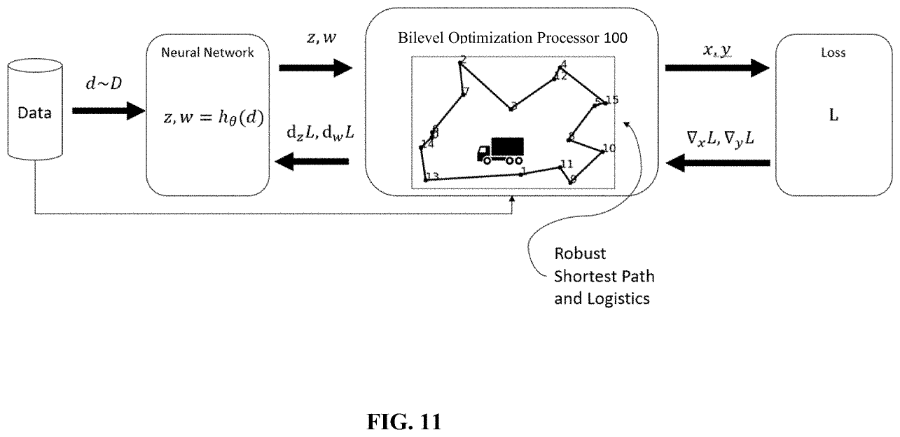

Other embodiments of the present invention may be applied in robust logistics applications, e.g., a robust vehicle routing problem. FIG. 11 illustrates an embodiment that solves a logistics application using bilevel optimization and a neural network.

In robust logistic, it may be a goal to assign vehicles and parcels and customers such that the solution is robust into changes in the environment. In a logistic system, the routing of a vehicle may be part of a more complex system. For instance, the gradient may be computed and input into a Logistic module. Then, the full system may be optimized in a way that is robust to changes in the environment, e.g. train cancellation or custom delay.

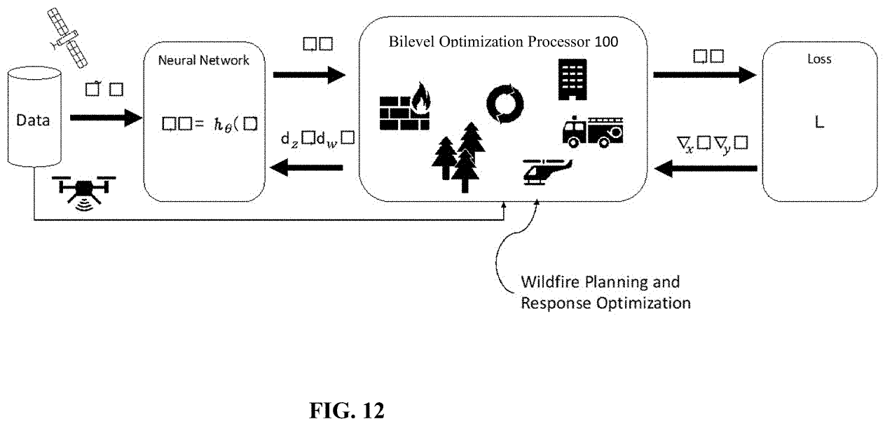

Another embodiment may be adapted for wildfire prevention and response or emergency management. FIG. 12 illustrates an embodiment for optimization of the response to natural or dangerous events using bilevel optimization and a neural network.

Emergency prevention and response may include the processing of real time and historical data from, e.g., a satellite, a ground survey, GIS (Geographical Information System), and reconnaissance from airborne vehicle and drones. A bilevel problem is used to describe the process of prevention and response against wildfires or natural or human caused events. Inputs may include: images at various spectral frequencies (e.g. images) of the area and feature describing the use and status of the land (carburant), historical events, extension of burns, presence of water sources, type of surface (water, rock, . . . ). The system can be trained on historical data and used to predict past dynamics to be able to predict the future and optimize the response.

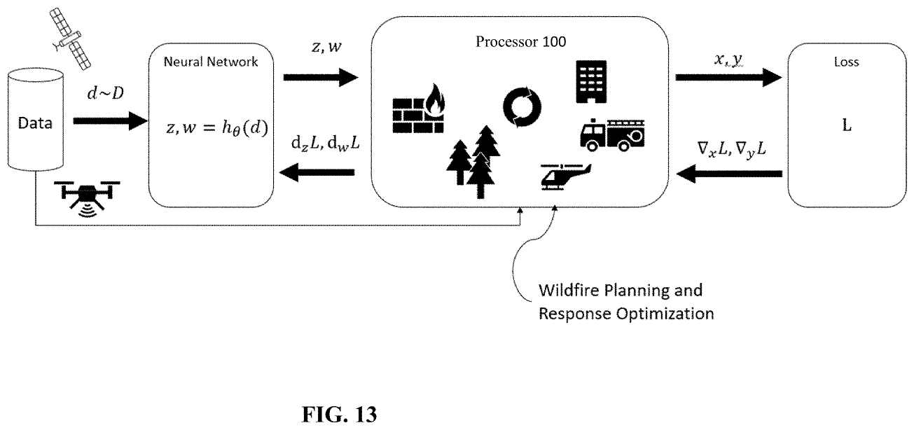

Embodiments of the present invention may also be adapted to industrial control and optimization. FIG. 13 illustrates an embodiment for industrial optimization using bilevel optimization and a neural network.

Industrial plants are composed by various systems where control actions are taken in response of the status of the plant. The bilevel problem may be used to describe the single process that builds the industrial plant thus optimize the whole system with end-to-end learning. For industrial application(s), the data may include and/or be the status of the plant: temperature, pressure, volume, flow at the various stage of the process, but also the status of elements that are processed. The action are described in term of variable of the model and can be the use of cooling system, device to stop or change the speed of fluids, increase or reduce the speed of processing.

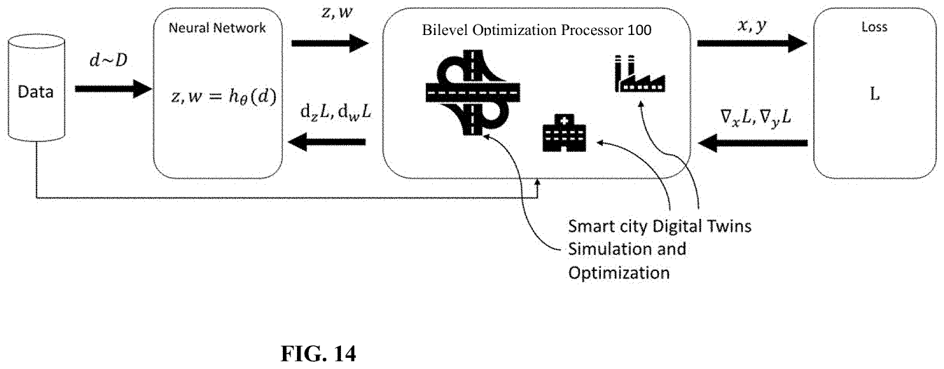

An embodiment of the present invention may be adapted for an application with digital twins for smart city and smart transportation. FIG. 14 illustrates an embodiment for smart transportation using bilevel optimization and a neural network.

A digital twin can be used to replicate the status of a system as, for example, the transport system, energy production plants, hospitals in term of the processes that participate for the provision of the service. For transport system this can be the number of vehicles or use of public services, as busses or trains. The control is implemented in term of policies parameterized by some quantities or by sophisticated control system as traffic control or traffic management services as parking directions or speed reduction or access restrictions. The interaction of the system with the traffic can be described with a bilevel problem and integrated into an end-to-end machine learning system.

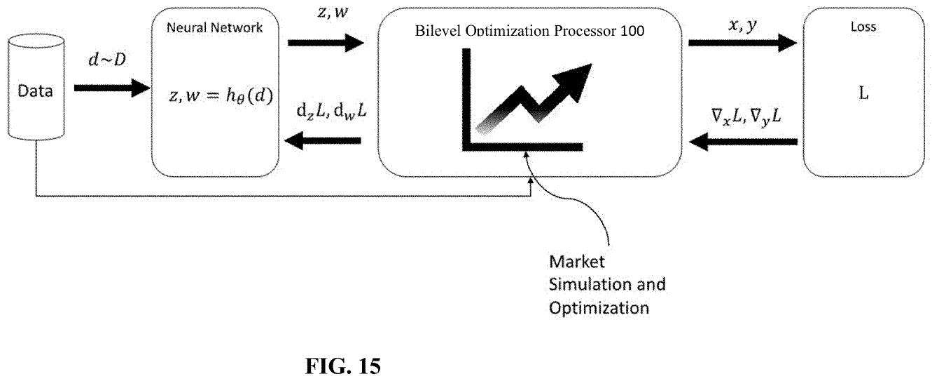

An embodiment of the present invention may be adapted for market price allocation and simulation. FIG. 15 illustrates an embodiment for market simulation using bilevel optimization and a neural network.

In a market simulation, it may be the case where it is a goal to analyze data from a market, where users buy quantity and other set the price. In this case, it can be assumed that there is an optimization problem that is solved by the market. It may be a goal to understand the hidden condition that generates the observational data, knowing that the market solves a bilevel optimization problem.

In this case, the observational data of the market is available, such as the prices and the sells. Also external information may be available. The model (e.g., the bilevel optimization processor 100 determined model) may then be used to predict and reproduce the past in order to predict the future.

An embodiment of the present invention includes separating the computation of the solution of the bilevel problem (using for example and existing solver) and the computations of the gradient around the current solution. The gradient can be evaluated by reformulating the gradient using implicit function and KKT conditions for the continuous case, and using the perturbation and argmax method for the discrete case.

Embodiments of the present invention provide at least the following advantages in contrast to current state of the art:
1. Allowing to integrate bilevel problems into a trainable architecture, as a module;
2. Using of automatic differentiation available in modern programming languages (PyTorch, JAX, autodiff);
3. Using of complex bilevel solvers without the requirement that those solvers been differentiable;
4. Efficient use of the VJP and JVP method of differentiation to avoid allocation of large memory;
5. Using discrete and continuous variables;
6. Allowing non-linear inequalities constraints and affine equality constraints; and
7. For the discrete case with multilinear function, making it possible to not compute the gradient.

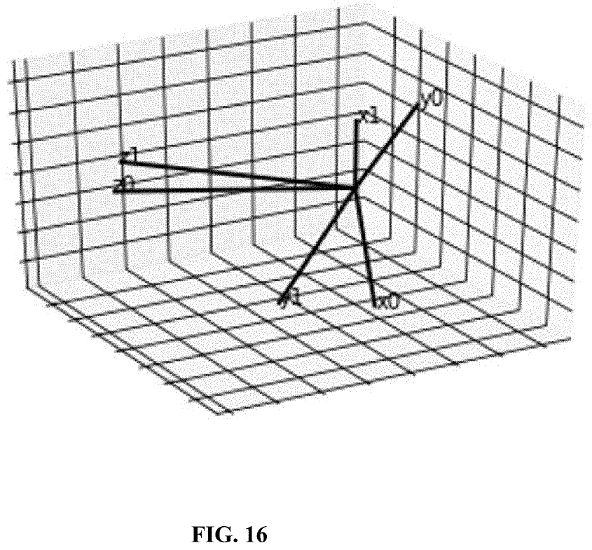

As an example, the problem of generation of orthogonal vectors in 3D (3 dimensions) may be considered. FIG. 16 illustrates an example of an initial configuration of orthogonal vectors in 3D.

The forward pass only requires to project new random vectors x and y, into the orthogonal space of the input vector z and vector x as the solution of connected optimization problems (bilevel problem).

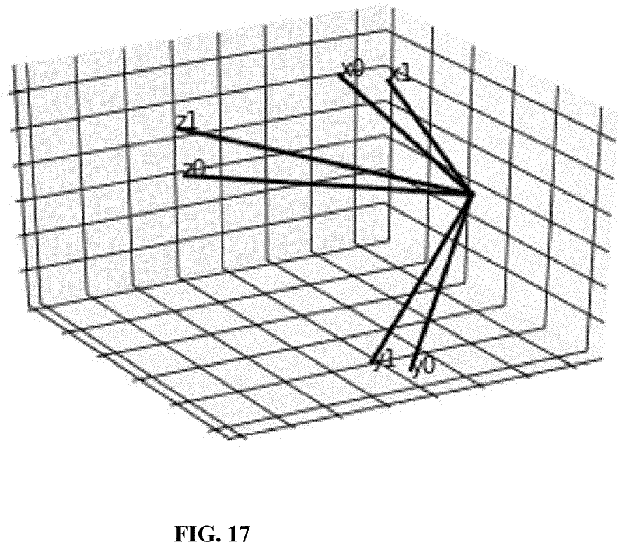

If the cost function is had on the output vectors x, y, this has no effect on the projector method. But this problem can be added into a differentiable system using an embodiment of the present invention, and training end-to-end. Now, the vectors are closely aligned with an error of circa 1%. FIG. 17 illustrates an output after allying an embodiment of the method of the present invention.

An embodiment of the present invention includes a method for integrating and training a bilevel problem inside a neural network system. The method comprises at least one of the following operations:
1. Collecting real-world input (data) and the problem description;
2. Computing a solution for the bilevel problem (e.g., using an existing solver) (find solution);
3. Computing with the gradient, which is propagated to the rest of the upstream neural network system (e.g., before the bilevel module/network on the left of the bilevel module) (compute changes);
4. Compute the update of the neural network (e.g., left) and update the parameters, including the one that defines the bilevel problem to be solve inside the network (implement/use changes);
5. Repeat operations 2 through 4 until convergence (e.g., until the change of parameters is small);
6. Overall output (parameters of the model) is a model to predict the output of the system in unknown input (e.g., the interaction of new proteins); and/or
7. Using the predictions of the model to design new systems (e.g., new drugs).

For instance, regarding operation 5 above, the change of the parameters and the gradient may be associated with each other. The relative change of the parameters may be measured to its value. For instance, convergence may occur when the difference is under 0.1% and this may be monitored based on the change in the x and y variables and/or only on the x variable (e.g., the outer variable).

In some examples, a method for solving bilevel optimization problems using machine learning/neural networks may be performed by a system comprising the bilevel optimization processor 100. For instance, the system may include, store, and/or update one or more neural networks (e.g., an upstream neural network and/or a downstream neural network) using the bilevel optimization processor 100 and/or one or more loss functions. The system may obtain data such as real-world input data. The data may be associated with the bilevel optimization problem. As mentioned above, the bilevel optimization problem may include two problems that are dependent on each other (e.g., a leader/outer problem and a follower/inner problem), which is known in the art. In some instances, the bilevel optimization problem may be associated with one or more embodiments described herein such as a digital twin for a smart hospital and/or a drug development problem. The data may be associated with real-world data for a hospital and/or drug development data. After obtaining the data, the system may input the data into the neural network to generate an output (e.g., the variable z). The output (e.g., the variable z) may indicate a cost value such as the weights of the neural network that specify the cost of the optimization problem.

The system, using the bilevel optimization processor 100, may train and update the neural network using one or more intermediate parameters associated with the neural network and the bilevel optimization problem. For instance, the bilevel optimization processor 100 may obtain the output (e.g., first output) of the neural network such as the variable z. The bilevel optimization processor 100 may further obtain a second output from a loss function associated with the neural network. Then, using the first and second outputs, the bilevel optimization processor 100 may determine one or more intermediate parameters that are used to update the neural network.

In some instances, the processor 100 may include sub-processors such as solution solver and a gradient solver. The first sub-processor (e.g., sub-processor 202) may be used to determine a solution (e.g., the variables x and y) for the bilevel optimization problem as described herein. The solution may be continuous variables and/or discrete variables. The sub-processor may use KKT reformation of the bilevel optimization problem and/or ADMM to determine the solution for the bilevel optimization problem. As mentioned above, the variable x indicates the outer level variables or the leader variables and the variable y indicates the solution of the follow or the inner problem that may be defined once the x variable is defined. For instance, in a hospital embodiment, the x variable may be the allocation of a patient to particular rooms and the y variable may be the cleaning of the operation room. In a drug development embodiment, the x variable may be the amount of protein injected into the cell while the variable y may be the process happening inside the cell. The x and y variables described herein are merely exemplary and may be related to any variables associated with bilevel optimization problems.

In some variations, the second sub-processor (e.g., sub-processor 204) may be used to update the neural network by determining one or more gradients based on the output (e.g., ∇ₓL, ∇_{y}L) from the loss function. These gradients (e.g., d_{z}L) may be used to update the neural network. The second sub-processor may determine the one or more gradients based on an implicit theorem of differentiability and/or Karush-Kuhn-Tucker (KKT) optimality conditions.

To put it another way, the processor 100 may update the neural network by determining one or more first intermediate parameters (e.g., y and x) based on the first output (e.g., z) from the neural network. Then, the processor 100 may input the one or more first intermediate parameters into the loss function to determine the second output (e.g., ∇ₓL, ∇_{y}L). Using the second output, the processor 100 may determine one or more second intermediate parameters (e.g., d_{z}L) associated with one or more gradients. Then, the processor 100 may provide the one or more second intermediate parameters to the neural network to update the neural network. In some examples, the one or more second intermediate parameters may be determined based on VJP or JVP.

The system may continue to update the neural network until it reaches convergence. For instance, the system may compare a change of the one or more intermediate parameters (e.g., y and z) to determine whether it has reached one or more pre-determined thresholds. Based on reaching the threshold, the system may output the one or more finalized parameters.

To put it another way, the system may continuously input new data into the neural network to train the neural network until it reaches a pre-determined threshold (e.g., a certain confidence interval). The output from the neural network, rather than being put directly into the loss function, may be adjusted, changed, and/or modified by the processor 100. For instance, rather than placing the output from the neural network into the loss function, the processor 100 may determine the intermediate parameters based on the output of the neural network and the intermediate parameters may be input into the loss function. In return, the processor 100 may receive one or more outputs from the loss function and determine one or more gradients based on the output. The processor 100 may compare the change in parameters to determine whether it has reached the pre-determined threshold. Based on reaching the pre-determined threshold, the system may generate the one or more finalized parameters using the one or more first or second intermediate parameters associated with a last iteration of updating the neural network. Based on not reaching the pre-determined threshold, the system may update the neural network again by determining new intermediate parameters (e.g., generating the first and second outputs as well as the first and second intermediate parameters).

The present disclosure also makes reference to Amos, Brandon, and J. Zico Kolter. “Optnet: Differentiable optimization as a layer in neural networks.” arXiv preprint arXiv:1703.00443 (2017) (the entire contents of which is hereby incorporated by reference herein).

Herein, the differentiation of a bilevel optimization problem is discussed.

In particular, among other contributions, the main contributions are multi-fold and include: use of bilevel problems as module and/or processor in a neural network architecture, derivation of the gradient with continuous variables, and/or derivation of the gradient with discrete variables.

Below, the problem of learning a model where a set of variables is a solution of a bilevel optimization problem is considered.

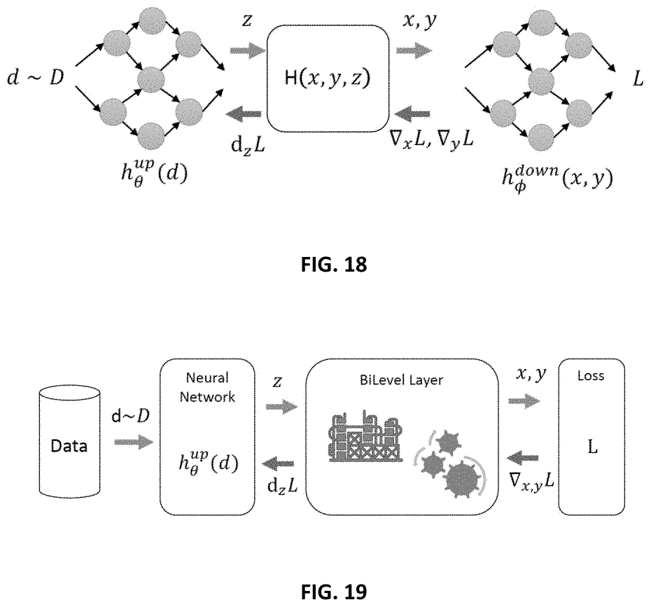

FIG. 18 shows an exemplary diagram for performing a task of learning a system, where x and y are the solutions of a bilevel optimization problem. In some instances, the below describes cases where either the variables are continuous or discrete, since, if they share the same motivation and structure, the approach may be different.

In particular, the system (e.g., a system comprising the processor 100) may have access to training data, D^{tr}={(d, u)ᵢ₌₁^{N}^{^{tr}}} and a loss function L(h_{ψ}∘H∘h_{θ}(d), u), where ∘ is the function composition operator, “d” is the input, and “u” is the output of the overall end-to-end learning system where the bilevel layer(s) is/are part of. The function H (x, y, z) defines the bilevel optimization problem, where once z is given, it is possible to compute the other variables x and y. FA solution may be assumed to exist and this solution is the minimal and is unique, i.e. (x, y)∈{x, y: H(x,y,z)=0} or (x, y) ∈ Solve(z).

At test time

$${D}^{te} = \{ {( d , u )}_{i = 1}^{{N}^{t e}} \} ,$$

the processor 100 may seek to predict the variable u, which underpins the solution of a bilevel optimization problem. Once access is obtained to the gradients for each layer, their parameters may be updated. While for the upstream and downstream functions, the gradients may be back-propagated using the chain rule, for the bilevel optimization layer this might not possible and in the case of discrete variables it might not even be well defined.

Referring to the above variables and equations, tr represents the training, to represents testing, D is the input data, N is the number of samples, L is the loss, h_{ψ} and h_{θ} are the upstream and downstream functions, and H is the function that defines the bilevel problem.

Physical System with control example: An example where a bilevel problem may be used is to model the interaction of a dynamical system and its control sub-system, as for example an industrial plant or a physical process.

In particular, FIG. 19 shows an example of an application to model a dynamical system with a control Neural Network where the interaction between the controller and the process is modelled as the solution of a Bilevel Optimization Problem. The control sub-system changes based on the state of the underlying dynamical system, which itself solves a physics constraint optimization problem (see e.g., Maziar Raissi, Paris Perdikaris, and George E Karniadakis. Physics-informed neural networks: A deep learning framework for solving forward and inverse problems involving nonlinear partial differential equations. Journal of Computational Physics, 378:686-707, 2019, and Filipe de Avila Belbute-Peres, Kevin Smith, Kelsey Allen, Josh Tenenbaum, and J Zico Kolter. End-to-end differentiable physics for learning and control. Advances in neural information processing systems, 31: 7178-7189, 2018, both of which are incorporated by reference herein). The inverse problem aims to estimate the parameters of the system from observational data, but the problem is complex when the control sub-system acts on the physical system.

Interdiction problem example: Two actors with Interdiction problems (see e.g., Matteo Fischetti, Ivana Ljubic, Michele Monaci, and Markus Sinnl. Interdiction games and monotonicity, with application to knapsack problems. INFORMS Journal on Computing, 31(2):390-410, 2019, which is incorporated by reference herein) arise in various areas, from marketing, protecting critical infrastructure, preventing drug smuggling to hindering nuclear weapon proliferation. This class of problem can be modelled naturally as a linear bilevel optimization problem, over discrete variables.

Min-max problem example: Min-max problems are used to model robust optimization problems (see e.g., Aharon Ben-Tal, Laurent El Ghaoui, and Arkadi Nemirovski. Robust optimization. Princeton university press, 2009, which is incorporated by reference herein), where a second variable represents the environment and is constrained to an uncertain set that captures the unknown variability of the environment.

Relationship to existing bilevel works: In previous works (see e.g., Luca Franceschi, Michele Donini, Paolo Frasconi, and Massimiliano Pontil. Forward and reverse gradient-based hyperparameter optimization. In International Conference on Machine Learning, pages 1165-1173. PMLR, 2017 and Benoît Colson, Patrice Marcotte, and Gilles Savard. An overview of bilevel optimization. Annals of operations research, 153(1):235-256, 2007, and Charles D Kolstad and Leon S Lasdon. Derivative evaluation and computational experience with large bilevel mathematical programs. Journal of optimization theory and applications, 65(3):485-499, 1990, all of which are incorporated by reference herein), the gradient (i.e. hyper-gradient) with respect to the outer-level problem variables of a bilevel optimization problem is used to solve the bilevel problem by unrolling the solution of the inner lever over few iterations (see e.g., Luca Franceschi, Michele Donini, Paolo Frasconi, and Massimiliano Pontil. Forward and reverse gradient-based hyperparameter optimization. In International Conference on Machine Learning, pages 1165-1173. PMLR, 2017), or derived its form for specific applications (see e.g., Aravind Rajeswaran, Chelsea Finn, Sham Kakade, and Sergey Levine. Meta-learning with implicit gradients. arXiv preprint arXiv:1909.04630, 2019 and Francesco Alesiani, Shujian Yu, Ammar Shaker, and Wenzhe Yin. Towards interpretable multi-task learning using bilevel programming. arXiv preprint arXiv:2009.05483, 2020, and Benoît Colson, Patrice Marcotte, and Gilles Savard. An overview of bilevel optimization. Annals of operations research, 153(1):235-256, 2007, all of which are incorporated by reference herein), while in other works the hyper-gradient is never explicitly computed, but the outer and inner problems are solved using nested loops, for example using Trust-region methods (see e.g., Benoît Colson, Patrice Marcotte, and Gilles Savard. An overview of bilevel optimization. Annals of operations research, 153(1):235-256, 2007). In these approaches, the hyper-gradient is not used to propagate the gradient from previous layers, thus it might not be known how to compute the gradient with respect to the problem parameters. In Matthew MacKay, Paul Vicol, Jon Lorraine, David Duvenaud, and Roger Grosse. Self-tuning networks: Bilevel optimization of hyperparameters using structured best-response functions. arXiv preprint arXiv:1903.03088, 2019, which is incorporated by reference herein, the authors propose a form for the best response function and use it to solve for x, y. It is not in the scope of these traditional approaches to propagate backwards the gradient information. Whereas in this application, the case where the solution of the bilevel optimization problem is the output (versus the input) and the gradient needs to flow from the downstream layers to upstream layers (back-propagation) is described and contemplated.

Embodiment(s) of application: By modeling the bilevel optimization problem as an implicit layer (see e.g., Shaojie Bai, J Zico Kolter, and Vladlen Koltun. Deep equilibrium models. arXiv preprint arXiv:1909.01377, 2019, which is incorporated by reference herein), a more general case is considered where 1) the solution of the bilevel problem is computed separately by a bilevel solver; thus leveraging on powerfully solver developed over various decades (see e.g., Thomas Kleinert, Martine Labbe, Ivana Ljubic, and Martin Schmidt. A survey on mixed-integer programming techniques in bilevel optimization. 2021, which is incorporated by reference herein); and 2) a gradient is estimated that is able to propagate the loss error to upstream layers from the gradients on downstream layers.

By separating the computation of the solution and the computation of the gradient around the current solution, the processor 100 may 1) compute the gradient more efficiently, in particular, the processor 100 may compute second order gradient taking advantage of the vector-jacobian product (push-back operator) formulation without explicitly inverting and thus building the jacobian or hessian matrices; 2) use more advanced and not differentiable solution techniques to solve the bilevel optimization problem that would be difficult to integrate using automatic differentiable operators.

In the following, the definition of bilevel problem is presented and it is shown how it is possible to determine the gradient with respect to the input features using the implicit function theorem and the Discrete Adjoin Method (DAM).

The Bilevel Programming is discussed below.

Two classes of problems are considered: 1) with continuous variables or 2) with discrete variable, i.e. where the set of values of variable is discrete. Both problem classes can be formalized as solving the following problem:

$${min}_{x ∈ X} f ( x , y , z ) \qquad ( 1 a )$$

$$y ∈ arg {min}_{y ∈ Y} g ( x , y , z ) \qquad ( 1 b )$$

The upper part of the problem is called outer optimization problem and resolves for the variable x∈X, where, for the continuous case, X= [symbol] ⁿ. When the outer problem variable is x alone is called pessimistic, while if it solves for (x, y) it is called optimistic, since the y variable helps in solving the upper problem. The second problem is called the inner optimization problem and solves for the variable y∈Y, with Y= [symbol] ^{m} with continuous variables. The variable z∈ [symbol] ^{p} is the input variable and is a parameter for the bilevel problem. It helps to think to x(z) and y(x, z) the solutions of the outer and inner problems. When the variable are discrete, the problem may be written differently, and in particular, the objective function may be restricted to be multi-linear or multi-affine. Various important combinatorial problems are linear in the discrete variables (e.g., vehicle routing problem (VRP), travelling salesman problem (TSP), Boolean satisfiability problem (SAT)), one example form is the following:

$${min}_{x ∈ X} {〈 z , x 〉}_{A} + {〈 y , x 〉}_{B} \qquad ( 2 a )$$

$$y ∈ arg {min}_{y ∈ Y} {〈 w , y 〉}_{C} + {〈 x , y 〉}_{D} \qquad ( 2 b )$$

The variables x, y have domains in ∈X, y∈Y, where X, Y are convex polytopes that are constructed from a set of distinct points χ⊂ [symbol] ⁿ, [symbol] ⊂ [symbol] ^{m}, as their convex hull. The outer and inner problems are Integer Linear Programs (ILPs). The extended inner product [symbol] x, y [symbol] _{A}=x^{T} Ay is considered to represent the multi-linear operator. This definition may be extend to the non linear case as σ( [symbol] x,y [symbol] _{A})=σ(x^{T} Ay). Separate parameter for the outer and inner problems, z∈ [symbol] ^{p} and w∈ [symbol] ^{q}, may be considered. An assumption may be made to either the solution to be unique or to be working in the neighbor of a local solution, thus the solution of the inner problem is considered unique. A special case of Bilevel optimization problem is the min-max:

$${min}_{y ∈ Y} {max}_{x ∈ X} f ( x , y , z ) \qquad ( 3 )$$

where the minimization function are equal and opposite in sign. Also in this case, with discrete variables, it may be assumed that ƒ (x, y, z) is a multi-linear function.

Gradient estimation for Bilevel Optimization Problem as Implicit Layer is discussed below.

Various practical problems can be model as bilevel optimization problem as in Eq. 1, either in the continuous or the discrete form. Even if the discrete and continuous variable cases share a similar structure, the approach is different when evaluating the gradients. The following basic steps may be identified: 1) In the forward pass, solve the combinatorial or continuous Bilevel Optimization problem as defined in Eq. 1 using existing solver; and 2) During the backward pass, compute the gradient d_{z}L (and d_{w}L) using the suggested gradients starting from the gradients on the output variables ∇ₓL and ∇_{y}L.

Regarding continuous variables, to evaluate the gradient of the variables z versus the loss function L, the gradients of the two output variables x, y through the two optimization problems may be propagated. The implicit function theorem may be used to approximate locally the function z→(x, y). Thus, the following main results may be determined.

Theorem 1. Let consider the problem defined in Eq. 1, then the total gradient of the parameter z with respect to the loss function L(x, y, z) is computed from the partial gradients ∇ₓL, ∇_{y} L, ∇_{z}L as

$${d}_{z} L = {∇}_{z} L - \vert \begin{matrix}{∇}_{x} L & {∇}_{y} L\end{matrix} \vert \begin{matrix}{∇}_{x} F \\ {∇}_{x} G\end{matrix} \begin{matrix} & {∇}_{y} F \\ & {∇}_{y} G\end{matrix} {\vert}^{- 1} \vert \begin{matrix}{∇}_{z} F \\ {∇}_{z} G\end{matrix} \vert \qquad ( 4 )$$

Theorem 2. Let consider the bilevel problem of Eq. 1, the following set of equations may be built that represent the equivalent problem around a given solution x*, y*, z*

F(x,y,z)=0 (5a)

G(x,y,z)=0 (5b)

where

F(x,y,z)=∇ₓf−∇_{y}f∇_{y}G∇ₓG (6a)

G(x,y,z)=∇_{y}g (6b)

where the short notation is used for f=f(x, y, z), g=g(x, y, z), F=F(x, y, z), G=G(x, y, z).

The implicit layer is thus defined by the two conditions F(x,y,z)=0 and G(x, y, z)=0. The Eq. 4 may be solved without explicitly compute the Jacobian matrices and inverting the system, but adopting the Vector-Jacobian product approach it can proceed from left to right to evaluate d_{z}L. In the following section, it is described how affine equality constraints and nonlinear inequality can be used when modelling f, g. It may also be noticed that the solution of Eq. 4 does not require to solve the original problem, but only to apply matrix-vector products, i.e. linear algebra, and the evaluation of the gradient that can be computed using automatic differentiation.

A first algorithm that describes the bilevel implicit layer is described below. For example, at the first step, there is an input: Training sample (\tilde{d}, ũ). The second step may include a forward pass such as: a) Compute (x, y)∈{x, y: H (x, y, z)=0} using Bilevel Solver (x, y)∈Solve (z); b) Compute the loss function L(h_{ψ}∘H∘h_{θ}(\tilde{d}),ũ); c) Save (x, y, z) for the backward pass. Then, a third step may include a backward pass such as: a) update the parameter of the downstream layers ψ using back-propagation; b) For the continuous variable case, compute based on Theorem 1 around the current solution (x, y, z), without solving the Bilevel Problem; c) For the discrete variable case, use the gradient estimates of Theorem 3, Section 3.2.1 or Section 3.2.2 by solving, when needed, for the two separate problems; d) Back-propagate the estimated gradient to the downstream parameters θ.

Regarding linear equality constraints, the model of Eq. 1 may be extended to the case where there are linear equality constraints on the two problems. The case may be:

Ax=b.

Then, the following variable substitution may be made:

x(r)=x₀+A^{⊥}r,

where A^{⊥} is the orthogonal space of A, i.e. A^{⊥}A=0, and x₀ is one solution of the equation, i.e. Aₓ_{ₒ}=b. Thus, a new variable r instead of x may be determined and a new function may be determined as:

ƒ(x,y,z)→ƒ(x₀+A^{⊥}x,y,z),g(x,y,z)→g(x₀+A^{⊥}x,y,z).

The same applies for the inner problem

Bx=c.

Then, the following substitution may be applied:

y(q)=y₀+B^{⊥}q,

where B^{⊥} is the orthogonal matrix of B and yₒ is such that B_{y}_{ₒ}=c, which induces a new function for the inner problem:

ƒ(x,y,z)→ƒ(x,y₀+B^{⊥}y,z),g(x,y,z)→g(x,y₀+B^{⊥}y,z).

Regarding non-linear inequality constraints, the model of Eq. 1 may be extended when there are non-linear inequality constraints. For example, the barrier method approach (see e.g., Stephen Boyd, Stephen P Boyd, and Lieven Vandenberghe. Convex optimization. Cambridge university press, 2004, which is incorporated by reference herein) may be used, where the variable is penalized with a logarithmic function to violate the constraints. Specifically, the case where ƒᵢ, gᵢ are inequality constraint functions, i.e. ƒᵢ<0, gᵢ<0 for the outer and inner problems may be considered, and then the new functions may be defined as:

$$f \to t f - {\sum }_{i = 1}^{{k}_{x}} ln ( - {f}_{i} ) ,$$

where t is a parameter that t→∞. The same approach can be applied to the inner problem, thus leading to a new unconstrained function

$$g \to t g - {\sum }_{i = 1}^{{k}_{y}} ln ( - {g}_{i} ) .$$

Regarding bilevel cone programming, the previous results may be applied also to the special case of cone programming (see e.g., Akshay Agrawal, Shane Barratt, Stephen Boyd, Enzo Busseti, and Walaa M Moursi. Differentiating through a cone program. arXiv preprint arXiv:1904.09043, 2019b, which is incorporated by reference herein), where the outer and inner optimization problems are restricted to solve cone programs. Cone programming includes convex programming (see e.g., Akshay Agrawal, Brandon Amos, Shane Barratt, Stephen Boyd, Steven Diamond, and Zico Kolter. Differentiable convex optimization layers. arXiv preprint arXiv:1910.12430, 2019a, which is incorporated by reference herein) and efficient solver for cone programs exists and can be used to compute a solution of the bilevel problem (see e.g., Aurelien Ouattara and Anil Aswani. Duality approach to bilevel programs with a convex lower level. In 2018 bilevel programs with a convex lower level. In 2018 1388-1395. IEEE, 2018).

$${min {c}^{T}}_{x} x + {( Cy )}^{T} x \qquad ( 7 a )$$

$$s . t . Ax + z + R ( y ) ( x - r ) = b \qquad ( 7 b )$$

$$s ∈ 𝒦 \qquad ( 7 c )$$

$$y ∈ arg {min}_{y} {d}^{T} y + {( Dx )}^{T} y \qquad ( 7 d )$$

$$s . t . By + u + P ( x ) ( y - p ) = f \qquad ( 7 e )$$

$$u ∈ 𝒦 \qquad ( 7 f )$$

In this bilevel cone programming, the inner and outer problem are both cone programs, where R(y), P(x) represents a linear transformation, while C, r, D, p are new parameters of the problem. Based on a local minima of Eq. 7 (e.g., local minima of 7a-7f) existing, an interior point method may be used to find such point. An assumption is made that this is a minimal point and that is not a saddle point. To compute the bilevel gradient, the residual maps (see e.g., Enzo Busseti, Walaa M Moursi, and Stephen Boyd. Solution refinement at regular points of conic problems. Computational Optimization and Applications, 74(3); 627-643, 2019, which is incorporated by reference herein) of the outer and inner problems may be used. Indeed, Theorem 1 may be applied, where F=N₁(x, Q, y) and G=N₂ (y, Q, x), and the normalized residual map defined in Busseti (see Enzo Busseti, Walaa M Moursi, and Stephen Boyd. Solution refinement at regular points of conic problems. Computational Optimization and Applications, 74(3); 627-643, 2019 and Akshay Agrawal, Brandon Amos, Shane Barratt, Stephen Boyd, Steven Diamond, and Zico Kolter. Differentiable convex optimization layers. arXiv preprintarXiv:1910.12430, 2019a) of the outer and inner problems.

Regarding discrete variables, the gradient might not be well defined. Thus, the present application may resort to estimate the gradients. For the bilevel problem with discrete variables, the present application may first consider the form Eq. 2. If the solution of the bilevel problem exists and its solution is given in Kleinert (see Thomas Kleinert, Martine Labbe, Ivana Ljubic, and Martin Schmidt. A survey on mixed-integer programming techniques in bilevel optimization. 2021, which is incorporated by reference herein) the partial gradients of the variables with respect to the input parameters assume the following form:

Theorem 3. Given the Eq. 2 problem, the partial variation of a cost function L(x, y, z, w) on the input parameters has the following form:

d_{z}L=∇_{z}L+[∇ₓL+∇_{y}L∇_{z}y]∇_{z}x (8a)

d_{w}L=∇_{w}L+[∇ₓL∇_{y}x+∇_{y}L]∇_{w}y (8b)

where

∇_{z}x(z,y)=A⁻¹∇_{z}_{²}²\tilde{ϕ}_{η}(z,y)|_{η→0} (9a)

∇_{w}y(w,z)=C⁻¹∇_{w}_{²}²\tilde{ψ}(w,z)|_{η→0} (91b)

∇ₓy(x,w)=D⁻¹∇ₓ_{²}²\tilde{Θ}_{η}(x,w)|_{η→0} (9c)

∇_{y}x(z,y)=B⁻¹∇_{y}_{²}²\tilde{W}_{η}(z,y)|_{η→0} (9d)

∇_{z}y=∇ₓy∇_{z}x (9e)

and

\tilde{ϕ}_{η}(z,y)= [symbol] _{u˜U}ϕ(z+ηu,y) (10a)

\tilde{ψ}_{η}(w,x)= [symbol] _{u˜U}ψ(w+ηu,x) (10b)

\tilde{Θ}_{η}(x,w)= [symbol] _{u˜U}ψ(w,x+ηu) (10c)

\tilde{W}_{η}(y,z)= [symbol] _{u˜U}ϕ(z,y+ηu) (10d),

while

$$ϕ ( z , y ) = {min}_{x ∈ X} {〈 z , x 〉}_{A} + {〈 y , x 〉}_{B} \qquad ( 11 a )$$

$$ψ ( w , x ) = {min}_{y ∈ Y} {〈 w , y 〉}_{C} + {〈 x , y 〉}_{D} \qquad ( 11 b )$$

under the condition of Berthet (see Quentin Berthet, Mathieu Blondel, Olivier Teboul, Marco Cuturi, Jean-Phillippe Vert, and Francis Bach. Learning with differentiable perturbed optimizers. arXiv preprint arXiv:2002.08676, 2020, which is incorporated by reference herein). Referring to the above, η is a user defined variable, [symbol] is the expected value, u is a variable that may also be defined by a user, and U is the distribution of u. Further, A, B, C, and D are matrices defined in the problem definition, which are fixed in this context. \tilde{ψ}_{η}, \tilde{W}_{η}, and \tilde{ϕ}_{η} are defined based on ϕ, ψ, and these are proxy functions to estimate the variable output (solution cost) after perturbation, which is shown in 11a and 11b above.

When the present application estimates the gradient by perturbing the input variable, the present application introduces large variance in the estimated quantities. The present application can see that this approach is similar to REINFORCE (see e.g., Ronald J Williams. Simple statistical gradient-following algorithms for connectionist reinforcement learning. Machine learning, 8(3-4):229-256, 1992), since the present application exchanges the expectation with the evaluation of the function, thus suffering similar effects. While it is possible to reduce variance in some cases (see e.g., Will Grathwohl, Dami Choi, Yuhuai Wu, Geoffrey Roeder, and David Duvenaud. Backpropagation through the void: Optimizing control variates for black-box gradient estimation. Arxiv preprint arXiv:1711.00123, 2017, which is incorporated by reference herein) with the use of additional trainable functions, the present application considers alternative approaches as described in the following.

One approach may include implicit differentiation by perturbation. For example, Domke, 2010 (see e.g., Justin Domke. Implicit differentiation by perturbation. Advances in neural Information Processing Systems, 23:523-531, 2010, which is incorporated by reference herein) shows how using implicit differentiation by perturbation can approximate

$${∇}_{z} L \approx \frac{1}{τ} [ x ( z + τ {Δ}_{x} L ) - x ( z ) ]$$

when x(z)=arg max_{x∈x}<x, z>+H(x) and H(x) is the entropy function introduced as regularization term. Using an alternative approach, Vlastelica et al., 2019 (see e.g., Marin Vlastelica Pogancic, Anselm Paulus, Vit Musil, Georg Martius, and Michal Rolinek. Differentiation of blackbox combinatorial solvers. In International Conference on Learning Representations, 2019, which is incorporated by reference herein) show a similar gradient estimator. The variation thus can be computed on the input variables from the two separate problems of the Bilevel Problem:

$${∇}_{Z} L \approx \frac{1}{τ} [ x ( z + τ {∇}_{x} L , y ) - x ( z , y ) ] \qquad ( 12 a )$$

$${∇}_{w} L \approx \frac{1}{τ} [ y ( w + τ {∇}_{y} L , x ) - y ( w , x ) ] \qquad ( 12 b )$$

where x(z, y) and y(w, x) represent the solutions of the two problems separately and τ→0 is a hyper-parameter. This form is more convenient of Eq. 8, since it does not require to compute the cross terms, ignoring thus the interaction of the two levels.

Another approach may be specific loss functions. In estimating the input variables z, w of the model, the present application may not be interested in the interaction between the two variable x, y. This for example happens, when the present application considers one or more specific cost functions, and may be extended in the general case, if a local change is considered.

The Hamming Loss function represents one of the loss functions. For example, the Hamming loss function is defined over binary variables as:

L^{H}(x,y)=L^{H}(x)+L^{H}(y)

where L^{H}(x)= [symbol] 1−x, x* [symbol] + [symbol] x, 1−x* [symbol] and x* is the true value. This loss counts the positions, where the two vectors differ. If ∇ₓL^{H}(x)=1−2x* is computed, then ∇ₓ_{ᵢ}L^{H}(x)=+1 if xᵢ*=0 and ∇ₓ_{ᵢ}L^{H}(x)=−1 if xᵢ*=1 may be determined. This information can be directly used to update the zᵢ variable in the linear term [symbol] z, x [symbol] , thus the gradients of the input variables can be estimated as ∇_{z}_{ᵢ}L^{H}=λ∇ₓ_{ᵢ}L^{H} and Δ_{w}_{ᵢ}L^{H}=λ∇_{y}_{ᵢ}L^{H}, with some weight λ<0. The intuition is that, when the weight zᵢ associated with the variable xᵢ is increased, the value of the variable xᵢ reduces.

The Regret Loss function represents another loss function. A stronger gradient estimator can be evaluated by considering the Regret loss function used in combinatorial optimization (see e.g., Adam N Elmachtoub and Paul Grigas. Smart “predict, then optimize”. arXiv preprint arXiv:1710.08005, 2017 and Jayanta Mandi and Tias Guns. Interior point solving for 1p-based prediction+ optimization. arXiv preprint arXiv:2010.13943, 2020, which are both incorporated by reference herein) problems or for the analysis of Online Learning algorithms

L^{R}(x,y)=L^{R}(x)+L^{R}(y)

where L^{R} (x)= [symbol] z*,x(z)−x* [symbol] with z*, x* the optimal cost and solution. In this case, computing the ∇ₓL^{R} (x)=z* and ∇_{y}L^{R}(y)=w* may reveal directly the optimal costs.

The below describes machine learning applications.

One particular machine learning application may include optimal control with adversarial disturbance.

For example, the design of a robust stochastic control for a Dynamical System may be considered. The problem is to find a feedback function u=ϕ(x) such that

$${{{min}_{ϕ} 𝔼 \frac{1}{T} {\sum }_{t = 0}^{T} ‖ {x}_{t} ‖}^{2} + ‖ ϕ ( {x}_{t} ) ‖}^{2} \qquad ( 13 a )$$

$$s . t . {x}_{t + 1} = A {x}_{t} + B ϕ ( {x}_{t} ) + {w}_{t} , ∀ t \qquad ( 13 b )$$

where xₜ ∈ [symbol] ⁿ is the state of the system, while wₜ is an independent and identically distributed (i.i.d.) random disturbance and x₀ is the initial state that it is either given or drawn from a known distribution. To solve this problem, the present application adopts the use of the Approximate dynamic programming (ADP) (see e.g., Yang Wang and Stephen Boyd. Fast evaluation of quadratic control-lyapunov policy. IEEE Transactions on Control Systems Technology, 19(4):939-946, 2010, which is incorporated by reference herein) that solves a proxy quadratic problem:

$${min}_{u} {u}^{T} P u + {x}_{t} {Q}_{u} + {q}^{t} u \qquad ( 14 a )$$

$$s . t . ‖ u {‖}_{2} \leq 1. \qquad ( 14 b )$$

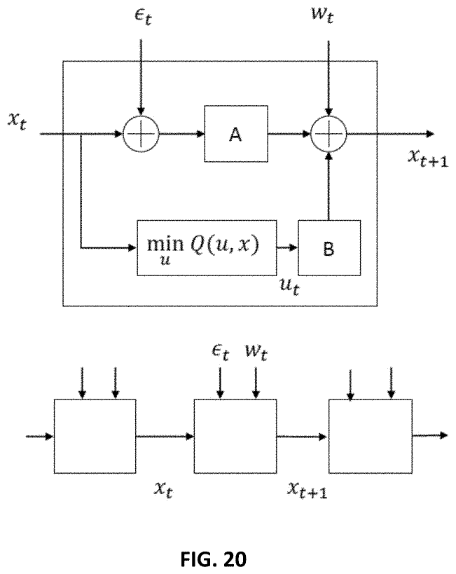

FIG. 20 shows a visualization of the Optimal Control Learning network, where a disturbance ϵₜ is injected based on the control signal uₜ.

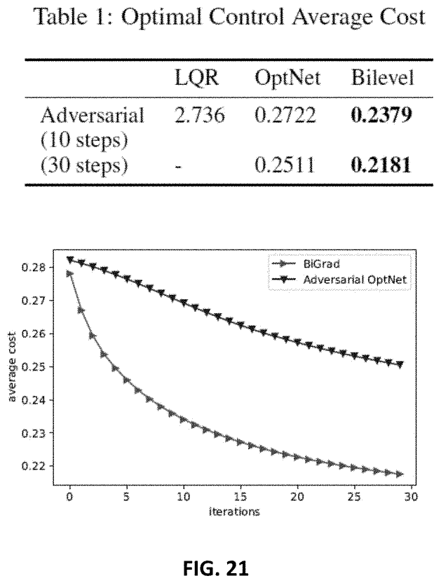

Further, the optimization layer may be used and propagate and/or update the problem variables (e.g. P; Q; q) using gradient descent. The bottom portion of FIG. 21 shows this. In particular, referring to FIG. 21, the top portion of FIG. 21 shows a table indicating the optimal control average cost. The bottom portion of FIG. 21 shows a comparison of the training performance for N=2, T=20 and epochs=10 of the BiGrad and the Adversarial version of the OptNet (see e.g., Brandon Amos and J Zico Kolter. Optnet: Differentiable optimization as a layer in neural networks. In International Conference on Machine Learning, pages 136-145. PMLR, 2017, which is incorporated by reference herein). The linear quadratic regulator (LQR) solution may be used as initial solution (see e.g., Rudolf Emil Kalman. When is a linear control system optimal? 1964, which is incorporated by reference herein). A resilient version of the controller may be built in the hypothesis that an adversarial is able to inject a noise of limited energy, but arbitrary dependent on the control u, by solving the following bilevel optimization problem

$${max}_{ϵ} Q ( u , x + ϵ ) \qquad ( 15 a )$$

$$s . t . ‖ ϵ ‖ \leq σ \qquad ( 15 b )$$

$$u ( ϵ ) = arg {min}_{u} Q ( u , x ) \qquad ( 15 c )$$

$$s . t . {‖ u ‖}_{2} \leq 1 \qquad ( 15 d )$$

where Q(u,x)=u^{T} Pu+xₜQu+q^{t}u.

The performance to verify the viability of the proposed approach can be compared with OptNet (see e.g., Brandon Amos and J Zico Kolter. Optnet: Differentiable optimization as a layer in neural networks. In International Conference on Machine Learning, pages 136-145. PMLR, 2017, which is incorporated by reference herein), where the outer problem is substituted with a best response function that computes the adversarial noise based on the computed output, in this case the adversarial noise is a scaled version of Qu of Eq. 14 (e.g., Eq. 14a and 14b).

The dynamic programming with the shortest path with interdiction is described below.

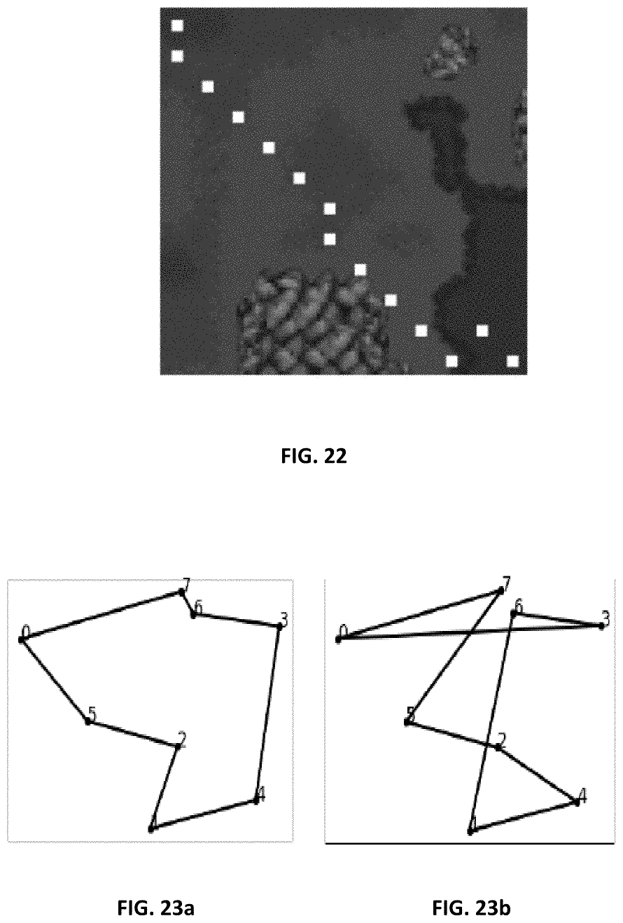

The problem of Shortest Path with Interdiction is considered, where the set of possible valid path is represented by Y, while the set of all possible interdiction is represented by X The mathematical problem can be written as

$${min}_{y ∈ Y} {max}_{x ∈ X} 〈 z + x ⊙ w , y 〉 \qquad ( 16 )$$

where ⊙ is the element wise product. This problem may be noticed as multi-linear in the x, y, z, which are discrete variables. In this specific case, the variables are associated with pixels of a WARCRAFT II map tile. FIG. 22 shows an example of the shortest path in the WARCRAFT II tile set of Guyomarch (see Jean Guyomarch. Warcraft ii open-source map editor, 2017. URL http://github.com/war2/war2edit., which is incorporated by reference herein). This was performed by closely following the scenario of Vlastelica et al. (see Marin Vlastelica Pogancic, Anselm Paulus, Vit Musil, Georg Martius, and Michal Rolinek. Differentiation of blackbox combinatorial solvers. In International Conference on Learning Representations, 2019), with the WARCRAFT II tile maps of Guyomarch. The interdiction game was implemented using a two stage min-max-min algorithm (see e.g., Nicolas Kammerling and Jannis Kurtz. Oracle-based algorithms for binary two-stage robust optimization. Computational optimization and Applications, 77(2); 539-569, 2020, which is incorporated by reference herein). In FIG. 23, it is possible to see the effect of interdiction on the final solution. It is also noted that the start and end positions will be always considered, since these are fixed in the embodiment. In other words, FIG. 23 shows an example of the shortest path without and with interdiction.

The combinatorial optimization embodiment such as a travel salesman problem (TSP) with interdiction is described below.

The Travel Salesman Problem (TSP) may include the input provided to the system is a subset of the total number of cities and an estimation of the distance among cities may be desired. The mathematical problem to solve is given by

$${min}_{y ∈ Y} {max}_{x ∈ X} 〈 z + x ⊙ w , y 〉 \qquad ( 17 )$$

where Y represents the set of valid tours, while X the valid interdiction, z, w are weight matrices, and y, x represent the actual tour and interdiction. Similar to the dynamic programming experiment, the interdiction game may be implemented using a two stage min-max-min algorithm (see e.g., Nicolas Kammerling and Jannis Kurtz. Oracle-based algorithms for binary two-stage robust optimization. Computational optimization and Applications, 77(2); 539-569, 2020, which is incorporated by reference herein). FIG. 23 shows the solution of a TSP problem and the interdiction solution with a single interdiction. For instance, FIG. 23 shows an example of TSP with 8 cities and the comparison of a TSP tour without (a) an interdiction or with (b) a single interdiction.

The proofs of Theorems 1-3 described above are provided below.

Proof of Theorem 1. In order to prove the theorem, the Discrete Adjoin Method (DAM) may be used. A cost function or functional L(x, y, z) evaluated at the output of our system may be considered. The present application and system is defined by the two equations F=0, G=0 from Theorem 2. First, consider the total variations: dL; dF=0, dG=

dL=∇ₓLdx+∇_{y}Ldy+∇_{z}Ldz

dF=∇ₓFdx+∇_{y}Fdy+∇_{z}Fdz

dL=∇ₓGdx+∇_{y}Gdy+∇_{z}Gdz

Next, consider dL+dFλ+dGγ=[∇ₓL+∇ₓFλ+∇ₓGγ]dx+[∇_{y}L+∇_{y}Fλ+∇_{y}Gγ]dy+[∇_{y}L+∇_{z}Fλ+∇_{z}Gγ]dz. Now, it may be asked

$${∇}_{x} L + {∇}_{x} F λ + {∇}_{x} G γ = 0 \qquad ( 18 )$$

$${∇}_{y} L + {∇}_{y} F λ + {∇}_{y} G γ = 0 \qquad ( 19 )$$

$$or$$

$$\vert \begin{matrix}{∇}_{x} F & & {∇}_{x} G \\ {∇}_{y} F & & {∇}_{y} F\end{matrix} \vert \vert \begin{matrix}λ \\ γ\end{matrix} \vert = - \vert \begin{matrix}{∇}_{x} L \\ {∇}_{y} L\end{matrix} \vert$$

Then, it may be computed that the d_{L}=Δ_{y}L+Δ_{z}Fλ+Δ_{z}Gγ with λ, γ from the previous equation.

Proof of Theorem 2. The second equation is derived by imposing the optimally condition on the inner problem. Since inequality and equality constraints might not be known, the optimal solution may equate the gradient with respect toy to zero, thus G=∇_{y}g=0. The first equation is also related to the optimality of the x variable with respect to the total derivative or hyper-gradient, thus the present application may have that 0=dₓƒ=∇ₓƒ+∇_{y} ƒ∇ₓy. In order to compute the variation of y, i.e. ∇ₓy, the implicit theorem may be applied to the inner problem, i.e. ∇ₓG+∇_{y}G∇ₓy=0, thus obtaining ∇ₓy=−∇_{y}⁻¹G∇ₓG.

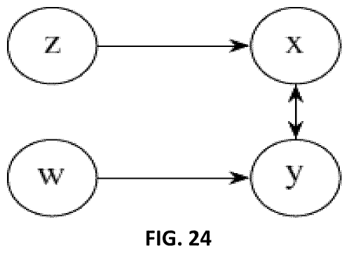

Proof of Theorem 3. The partial derivatives are obtained by using the perturbed minimum problem of the discrete problems defined by Eqs. 11a and 11b. It may be first notice that ∇ₓmin_{y∈Y}<x,y>=arg min_{y∈Y}<x,y>. This result is obtained by the fact that min_{y∈Y}<x,y>=<x,y*>, where y*=arg min_{y∈y}<x,y> and applying the gradient w.r.t. the continuous variable x. Eqs. 10a-10d represent the perturbed minimized. Thus, if the gradient of the perturbed minimizer is computed, the optimal solution is obtained, proper scaled by the inner product matrix. For example, ∇ₓ\tilde{ϕ}_{η}=Ax*(z, y), with A the inner product matrix. To compute the variation on the two parameter variables, it may be determined that dL=∇ₓLdx+∇_{y}Ldy+∇_{z}Ldz+∇_{w}Ldw and that dw/dz=0, dz/dw=0 from the dependence diagram of FIG. 24. FIG. 24 shows the Discrete Bilevel Variables and Dependence diagram.

While embodiments of the invention have been illustrated and described in detail in the drawings and foregoing description, such illustration and description are to be considered illustrative or exemplary and not restrictive. It will be understood that changes and modifications may be made by those of ordinary skill within the scope of the description. In particular, the present invention covers further embodiments with any combination of features from different embodiments described. Additionally, statements made herein characterizing the invention refer to an embodiment of the invention and not necessarily all embodiments.

The terms used in the disclosure should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. A method for bilevel optimization using machine learning, the method comprising:
obtaining input data associated with the bilevel optimization;
determining a solution for a bilevel problem;
updating, based on the solution for the bilevel problem, a neural network using one or more intermediate parameters associated with the neural network and the bilevel optimization, wherein the one or more intermediate parameters are based on first output from the neural network and second output from a loss function associated with the neural network, wherein the first output is generated based on inputting the input data into the neural network; and
outputting one or more finalized parameters for the bilevel optimization based on a change of the one or more intermediate parameters reaching a pre-determined threshold.

2. The method according to claim 1, wherein determining the solution for the bilevel problem is based on using Karush-Kuhn-Tucker (KKT) reformulation of the bilevel problem and/or alternating direction method of multipliers (ADMM).

3. The method according to claim 1, wherein updating the neural network using the one or more intermediate parameters comprises:
determining one or more gradients based on the second output from the loss function; and
updating the neural network based on providing the one or more gradients to the neural network.

4. The method according to claim 3, wherein determining the one or more gradients is further based on an implicit theorem of differentiability and/or Karush-Kuhn-Tucker (KKT) optimality conditions.

5. The method according to claim 1, wherein determining the solution for the bilevel problem comprises determining one or more first intermediate parameters to the bilevel optimization based on the first output from the neural network, and wherein updating the neural network using the one or more intermediate parameters comprises:
inputting the one or more first intermediate parameters into the loss function to determine the second output;
determining one or more second intermediate parameters based on the second output, wherein the one or more second intermediate parameters are associated with one or more gradients; and
providing the one or more second intermediate parameters to the neural network to update the neural network.

6. The method according to claim 5, further comprising:
repeatedly determining the one or more first intermediate parameters and updating the neural network using the one or more intermediate parameters;
after each iteration of updating the neural network, comparing a change of the one or more intermediate parameters with the pre-determined threshold; and
based on the change of the one or more intermediate parameters reaching the pre-determined threshold, generating the one or more finalized parameters using the one or more first or second intermediate parameters associated with a last iteration of updating the neural network.

7. The method according to claim 6, wherein repeatedly determining the one or more first intermediate parameters and updating the neural network using the one or more intermediate parameters is based on the change of the one or more intermediate parameters not reaching the pre-determined threshold.

8. The method according to claim 5, wherein the one or more first intermediate parameters to the bilevel optimization are continuous variables.

9. The method according to claim 5, wherein the one or more first intermediate parameters to the bilevel optimization are discrete variables.

10. The method according to claim 5, wherein determining the one or more second intermediate parameters associated with the one or more gradients is based on vector jacobian product (VJP) or jacobian vector product (JVP).

11. The method according to claim 1, wherein the bilevel problem is a hospital digital twin of a smart hospital, and wherein the method further comprises:
using the one or more finalized parameters to design a new system for the bilevel problem, wherein the new system optimizes patient scheduling for the smart hospital, room scheduling for the smart hospital, personnel scheduling for the smart hospital, recovery dynamic for the smart hospital, or procurement for the smart hospital.

12. The method according to claim 1, wherein the bilevel problem is a drug development problem, and wherein the method further comprises:
using the one or more finalized parameters to design a new system for the bilevel problem, wherein the new system indicates drug candidates for molecule synthesis.

13. A system comprising one or more processors which, alone or in combination, are configured to provide for execution of a method comprising:
obtaining input data associated with bilevel optimization;
determining a solution for a bilevel problem;
updating, based on the solution for the bilevel problem, a neural network using one or more intermediate parameters associated with the neural network and the bilevel optimization, wherein the one or more intermediate parameters are based on first output from the neural network and second output from a loss function associated with the neural network, wherein the first output is generated based on inputting the input data into the neural network; and
outputting one or more finalized parameters for the bilevel optimization based on a change of the one or more intermediate parameters reaching a pre-determined threshold.

14. The system of claim 13, wherein updating the neural network using the one or more intermediate parameters comprises:
determining one or more gradients based on the second output from the loss function; and
updating the neural network based on providing the one or more gradients to the neural network.

15. A tangible, non-transitory computer-readable medium having instructions thereon which, upon being executed by one or more processors, alone or in combination, provide for execution of a method comprising:
obtaining input data associated with a bilevel optimization;
determining a solution for a bilevel problem;
updating, based on the solution for the bilevel problem, a neural network using one or more intermediate parameters associated with the neural network and the bilevel optimization, wherein the one or more intermediate parameters are based on first output from the neural network and second output from a loss function associated with the neural network, wherein the first output is generated based on inputting the input data into the neural network; and
outputting one or more finalized parameters for the bilevel optimization based on a change of the one or more intermediate parameters reaching a pre-determined threshold.
